(19) United States
(12) Patent Application Publication
(10) Pub. No.: US 2022/0313601 A1
(43) Pub. Date: Oct. 6, 2022

(54) APPARATUS AND METHOD FOR FORMING NICOTINE GUM

(71) Applicant: Altria Client Services LLC, Richmond, VA (US)

(72) Inventors: Dwight David WILLIAMS, Powhatan, VA (US); David PHILLIPS, Richmond, VA (US); Feng GAO, Midlothian, VA (US); Anthony KIEU, Richmond, VA (US)

(73) Assignee: Altria Client Services LLC, Richmond, VA (US)

(21) Appl. No.: 17/223,415

(22) Filed: Apr. 6, 2021

(51) Int. Cl. A61K 9/68; A61K 31/465; A23G 4/04; A23P 30/10; A23G 4/12; B30B 9/28

(52) U.S. Cl. CPC A61K 9/0058; A61K 31/465; A23G 4/04; A23P 30/10; A23G 4/126; B30B 9/28; A23V 2002/00

(57) ABSTRACT

An apparatus for forming a nicotine gum may include a first pair of drums configured to receive and press a nicotine gum mixture to a first thickness so as to form a first pressed sheet and a second pair of drums configured to receive and score the first pressed sheet so as to form a scored sheet including a plurality of gum pieces. The second pair of drums may include a first drum having a first die with a first pattern and a second drum having a second die with a second pattern. The second pattern may be a mirror image of the first pattern. The first drum and the second drum may be spaced such that as the first pressed sheet passes between the first drum and the second drum, the first die and the second die at least score the first pressed sheet.

### BACKGROUND

### Field

At least one example embodiment relates to an apparatus and method for forming gum, such as a nicotine gum.

### Description of Related Art

Oral nicotine products are available in a variety of formats, such as chewing gums, sprays, lozenges, dissolvable tablets, non-dissolvable chews, films, gels, capsules, and pouches (e.g., containing fibers or granules). Oral products may have nicotine levels that create a familiar experience for adult tobacco consumers.

### SUMMARY

At least some example embodiments relate to an apparatus for forming a nicotine gum.

In at least one example embodiment, the apparatus may include a first pair of drums and a second pair of drums. The first pair of drums may be configured to receive and press a nicotine gum mixture to a first thickness so as to form a first pressed sheet. The second pair of drums may be configured to receive and score the first pressed sheet so as to form a scored sheet including a plurality of gum pieces. The second pair of drums may include a first drum and a second drum, where the first drum includes a first die having a first pattern, and the second drum includes a second die having a second pattern. The second pattern may be a mirror image of the first pattern. The first drum and the second drum may be spaced such that as the first pressed sheet passes between the first drum and the second drum, the first die and the second die at least score the first pressed sheet.

In at least one example embodiment, the first die may define a first plurality of recessed portions.

In at least one example embodiment, the second die may define a second plurality of recessed portions corresponding to the first plurality of recessed portions.

In at least one example embodiment, the second pair of drums may be configured to align a first plurality of non-recessed portions on the first die with a second plurality of non-recessed portions on the second die so as to at least score the first pressed sheet into the plurality of gum pieces.

In at least one example embodiment, the first drum of the second pair of drums may be configured to rotate at a first speed. The second drum of the second pair of drums may be configured to rotate at a second speed. The first speed and the second speed may be established to facilitate consistent material transfer. When a first plurality of non-recessed portions on the first die is aligned with a second plurality of non-recessed portions on the second die, the non-recessed portions at least score the first pressed sheet.

In at least one example embodiment, each of the first plurality of recessed portions may have a triangular cross-section along an upper surface of a non-recessed portion of the first die.

In at least one example embodiment, a first segment of a non-recessed portion on the first die may define one side of each of a pair of adjacent recessed portions.

In at least one example embodiment, the second pair of drums may be downstream of the first pair of drums.

In at least one example embodiment, a third pair of drums may be between the second pair of drums and the first pair of drums. The third pair of drums may be configured to receive and press the first pressed sheet to a second uniform thickness.

In at least one example embodiment, a heater may be configured to heat the first drum of the second pair of drums, the second drum of the second pair of drums, or a combination thereof, so as to heat the first pressed sheet.

At least some example embodiments relate to a method for forming a nicotine gum.

In at least one example embodiment, the method for forming the nicotine gum may include passing a sheet of a gum mixture through a first pair of drums; pressing the sheet between the first pair of drums to a first thickness so as to form a first pressed sheet; and passing the first pressed sheet through a second pair of drums. A first drum of the second pair of drums may include a first die, and a second drum of the second pair of drums may include a second die. The first die may include a first pattern of recessed portions, and the second die may include a second pattern of recessed portions. The first pattern of recessed portions may be a mirror image of the second pattern of recessed portions, so as to at least score the first pressed sheet and form a scored sheet including a plurality of gum pieces.

In at least one example embodiment, the second pair of drums may be downstream of the first pair of drums.

In at least one example embodiment, the method may further include passing the first pressed sheet through a third pair of drums. The third pair of drums may be between the second pair of drums and the first pair of drums, so as to press the first pressed sheet to a second uniform thickness.

In at least one example embodiment, the second uniform thickness may range from 2 mm to 18 mm.

In at least one example embodiment, the method may further include regulating a temperature of the first pair of drums and the second pair of drums.

In at least one example embodiment, regulating a temperature of the first pair of drums and the second pair of drums may include heating the second pair of drums so as to increase the temperature of the first pressed sheet.

In at least one example embodiment, regulating a temperature of the first pair of drums and the second pair of drums may include cooling the second pair of drums so as to reduce the temperature of the first pressed sheet.

In at least one example embodiment, passing the first pressed sheet through a second pair of drums may include aligning a first non-recessed portion on the first die with a second non-recessed portion on the second die so as to at least score the first pressed sheet.

In at least one example embodiment, the first thickness may range from 4 mm to 20 mm.

In at least one example embodiment, passing the first pressed sheet through a second pair of drums may include rotating the first drum of the second pair of drums at a first speed; rotating the second drum of the second pair of drums at a second speed, the first speed being the same as the second speed; and aligning a non-recessed portion of the first pattern of recessed portions on the first die with a non-recessed portion of the second pattern of recessed portions on the second die so as to at least score the first pressed sheet and form the plurality of gum pieces.

At least some example embodiments relate to an apparatus for forming a nicotine gum.

In at least one example embodiment, the apparatus for forming a nicotine gum includes a first pair of drums and a second pair of drums. The first pair of drums may be configured to receive and press a nicotine gum mixture to a first thickness so as to form a first pressed sheet. The second pair of drums may be configured to receive and cut the first pressed sheet so as to form a plurality of gum pieces. The second pair of drums may include a first drum and a second drum. The first drum may include a first die having a first pattern. The second drum may include a second die having a second pattern. The second pattern may be a mirror image of the first pattern. The first drum and the second drum may be spaced such that as the first pressed sheet passes between the first drum and the second drum, the first die and the second die cut the plurality of gum pieces.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a perspective view of a first side of an apparatus for forming nicotine gum according to at least one example embodiment.

FIG. 2 is a perspective view of a second side of an apparatus for forming nicotine gum according to at least one example embodiment.

FIG. 3 is a schematic view of an apparatus for forming nicotine gum according to at least one example embodiment.

FIG. 4 is an end perspective view of an apparatus for forming nicotine gum according to at least one example embodiment.

FIG. 5 is a perspective view of a die used in an apparatus for forming nicotine gum according to at least one example embodiment.

FIG. 6 is a front view of a die used in an apparatus for forming nicotine gum according to at least one example embodiment.

FIG. 7 is a side view of a die used in an apparatus for forming nicotine gum according to at least one example embodiment.

FIG. 8A is a top view of a first pressed sheet according to at least one example embodiment.

FIG. 8B is a side view of a first pressed sheet according to at least one example embodiment.

FIG. 9A is a top view of a second pressed sheet according to at least one example embodiment.

FIG. 9B is a side view of a second pressed sheet according to at least one example embodiment.

FIG. 10A is a top view of a scored sheet according to at least one example embodiment.

FIG. 10B is a side view of a scored sheet according to at least one example embodiment.

FIG. 11A is a cross-sectional view of a recessed portion of a die for forming gum pieces having a triangular cross-section along an upper surface of a non-recessed portion according to at least one example embodiment.

FIG. 11B is a cross-sectional view of a recessed portion of a die for forming gum pieces having a cross-section through a thickness and bisecting a vertex according to at least one example embodiment.

FIG. 11C is a cross-sectional view of a die cut along a longitudinal axis of the die and having recessed portions bisected through a vertex thereof according to at least one example embodiment.

FIG. 11D is a cross-sectional view of a die cut along an axis orthogonal to a longitudinal axis of the die and having recessed portions according to at least one example embodiment.

FIG. 12A is a cross-sectional view of a recessed portion of a die along an upper surface of a non-recessed portion for forming gum pieces having a square cross-section according to at least one example embodiment.

FIG. 12B is a cross-sectional view of a recessed portion of a die for gum pieces having a cross-section through a thickness according to at least one example embodiment.

FIG. 13A is a cross-sectional view of a recessed portion of a die along an upper surface of a non-recessed portion for forming gum pieces having a rectangular cross-section according to at least one example embodiment.

FIG. 13B is a cross-sectional view of a recessed portion of a die for forming gum pieces having a cross-section through a thickness according to at least one example embodiment.

FIG. 14A is a cross-sectional view of a recessed portion of a die along an upper surface of a non-recessed portion for forming gum pieces having a circular cross-section according to at least one example embodiment.

FIG. 14B is a cross-sectional view of a recessed portion of a die for forming gum pieces having a cross-section through a thickness according to at least one example embodiment.

FIG. 15A is a cross-sectional view of a recessed portion of a die along an upper surface of a non-recessed portion for forming gum pieces having a trapezoidal cross-section according to at least one example embodiment.

FIG. 15B is a cross-sectional view of a recessed portion of a die for forming gum pieces having a cross-section through a thickness according to at least one example embodiment.

FIG. 16A is a cross-sectional view of a recessed portion of a die along an upper surface of a non-recessed portion for forming gum pieces having a stadium cross-section according to at least one example embodiment.

FIG. 16B is a cross-sectional view of a recessed portion of a die for forming gum pieces having a cross-section through a thickness according to at least one example embodiment.

FIG. 17 is a perspective view of a die used in an apparatus for forming nicotine gum according to at least one example embodiment.

FIG. 18 is a front view of a die used in an apparatus for forming nicotine gum according to at least one example embodiment.

FIG. 19 is a schematic view of an apparatus for forming nicotine gum according to at least one example embodiment.

FIG. 20 is a schematic diagram of a control system for an apparatus for forming nicotine gum according to at least one example embodiment.

FIG. 21 is a flow chart of a method of forming nicotine gum according to at least one example embodiment.

### DETAILED DESCRIPTION OF EXAMPLE EMBODIMENTS

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” “attached to,” “adjacent to,” or “covering” another element or layer, it may be directly on, connected to, coupled to, attached to, adjacent to or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, region, layer, or section discussed below could be termed a second element, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, and/or elements, but do not preclude the presence or addition of one or more other features, integers, steps, operations, elements and/or groups thereof.

When the words “about” and “substantially” are used in this specification in connection with a numerical value, it is intended that the associated numerical value include a tolerance of ±10% around the stated numerical value, unless otherwise explicitly defined. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

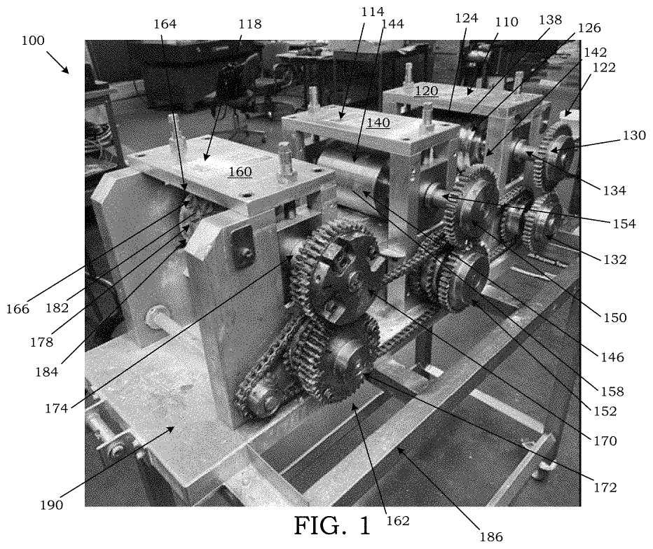

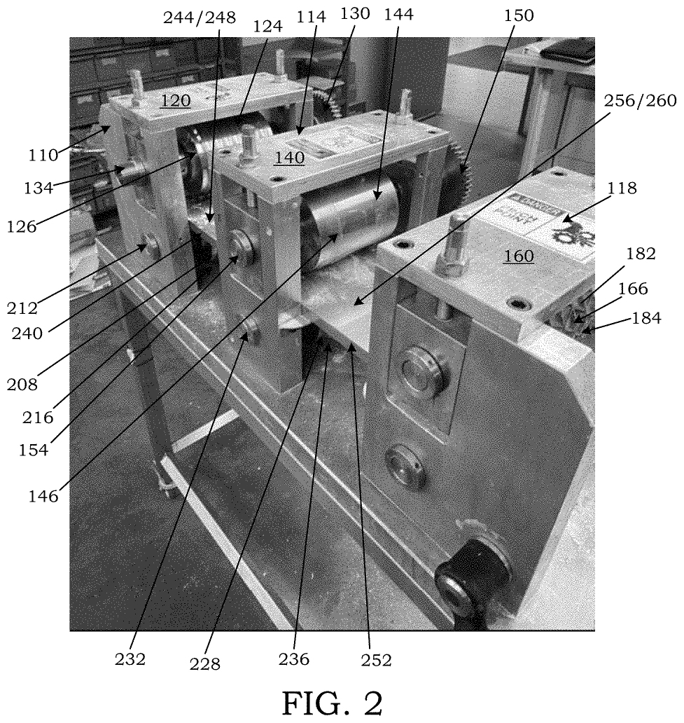

FIG. 1 is a perspective view of a first side of an apparatus for forming nicotine gum according to at least one example embodiment; and FIG. 2 is a perspective view of a second side of an apparatus for forming nicotine gum according to at least one example embodiment.

In at least one example embodiment, an apparatus 100 configured to form nicotine gum includes a first section 110, a second section 114, and a third section 118. In at least one example embodiment, the first section 110, second section 114, and third section 118 process a gum mixture to form a plurality of gum pieces.

In at least one example embodiment, the gum mixture is a nicotine gum mixture including a gum base polymer, an oil, and nicotine or a nicotine derivative. In at least one example embodiment, the gum base polymer includes polyvinyl acetate (PVA) and the oil includes a triglyceride. In at least one example embodiment, the nicotine gum mixture may be the nicotine gum mixture described in U.S. patent application Ser. No. ______, filed concurrently herewith on Apr. 6, 2021, titled “CONTROLLED-RELEASE NICOTINE CHEWING GUM,” Attorney Docket No. 24000NV-000733-US, the disclosure of which is incorporated herein in its entirety by reference. While a nicotine gum mixture is described, it is understood that the disclosure is not limited thereto. In at least one alternative example embodiment, the gum mixture is a cannabinoid gum mixture, a non-nicotine gum mixture, or any suitable gum mixture.

In at least one example embodiment, the apparatus 100 may process the gum mixture (hereinafter described with respect to, but not limited to, a nicotine gum mixture) to at least partially form a plurality of gum pieces where each of the plurality of gum pieces is sized and shaped to be wholly received in an oral cavity of an adult tobacco consumer.

Referring to FIGS. 1 and 2, in at least one example embodiment, the first section 110 includes a first tower having a first housing 120, a first pair of gear assemblies 122, and a first pair of drums 124. The first pair of drums includes a first upper drum 126 and a first lower drum 208 (see FIG. 2). The first housing 120 at least partially encloses the first upper drum 126 and the first lower drum 208. The first upper drum 126 is positioned adjacent to, and vertically above, the first lower drum 208. In at least one example embodiment, the first upper drum 126 and the first lower drum 208 are cylindrical drums. The first upper drum 126 and the first lower drum 208 may rotate within the first housing 120 to process the gum mixture.

In at least one example embodiment, the first pair of gear assemblies 122 includes a first upper gear assembly 130 and a first lower gear assembly 132. The first upper drum 126 is rotated by the first upper gear assembly 130, which is outside of the first housing 120. The first upper gear assembly 130 includes one or more wheels. Each of the one or more wheels includes teeth which intermesh to transfer movement from one wheel to another.

In at least one example embodiment, the first upper gear assembly 130 is connected to the first upper drum 126 by a first upper shaft 134, fixing the first upper drum 126 for rotation by the first upper gear assembly 130. In at least one example embodiment, the first upper gear assembly 130 is rotated, either manually or by a motor, as described below, to rotate the first upper drum 126.

In at least one example embodiment, the first lower drum 208 is rotated by the first lower gear assembly 132, which is outside of the first housing 120. The first lower gear assembly 132 may include one or more wheels having teeth which intermesh to transfer movement from one wheel to another.

The first lower gear assembly 132 is connected to the first lower drum 208 by a first lower shaft 212 (see FIG. 2), fixing the first lower drum 208 for rotation by the first lower gear assembly 132. In at least one example embodiment, the first upper gear assembly 130 engages with the first lower gear assembly 132 to either drive or be driven by rotation of the first lower gear assembly 132. The first upper gear assembly 130 and the first lower gear assembly 132 determine the speed relationship between the first upper drum 126 and the first lower drum 208.

In at least one example embodiment, the first upper drum 126 and the first lower drum 208 are first pressing drums. In at least one example embodiment, the first upper drum 126 includes a smooth outer surface 138 and the first lower drum 208 includes a smooth outer surface 216 for contacting the gum mixture. In other example embodiments, the outer surface 138 of the first upper drum 126 and the outer surface 216 of the first lower drum 208 may be textured (not shown). In other example embodiments, the outer surfaces 138, 216 may each include a non-stick coating (not shown). The first pair of drums 124 may receive the gum mixture, and as the first upper drum 126 and first lower drum 208 rotate, the first upper drum 126 presses the gum mixture against the first lower drum 208 to form the gum mixture into a pressed sheet having a first thickness, as further described below.

In at least one example embodiment, the second section 114 includes a second tower having a second housing 140, a second pair of gear assemblies 142, and a second pair of drums 144. The second pair of drums 144 includes a second upper drum 146 and a second lower drum 228 (see FIG. 2). The second housing 140 may at least partially enclose the second upper drum 146 and the second lower drum 228. The second upper drum 146 is positioned adjacent to, and vertically above, the second lower drum 228. In at least one example embodiment, the second upper drum 146 and the second lower drum 228 are cylindrical drums. The second upper drum 146 and the second lower drum 228 may rotate within the second housing 140 to collectively process the first pressed sheet.

In at least one example embodiment, the second pair of gear assemblies 142 includes a second upper gear assembly 150 and a second lower gear assembly 152. The second upper drum 146 is rotated by the second upper gear assembly 150, which is outside of the second housing 140. The second upper gear assembly 150 may include one or more wheels having teeth which intermesh to transfer movement from one wheel to another.

In at least one example embodiment, the second upper gear assembly 150 is connected to the second upper drum 146 by a second shaft 154, fixing the second upper drum 146 for rotation by the second upper gear assembly 150. In at least one example embodiment, the second upper gear assembly 150 is rotated, either manually or by a motor, as described below, to rotate the second upper drum 146.

In at least one example embodiment, the second lower drum 228 is rotated by the second lower gear assembly 152, which is outside of the second housing 140. The second lower gear assembly 152 may include one or more wheels having teeth which intermesh to transfer movement from one wheel to another.

In at least one example embodiment, the second lower gear assembly 152 is connected to the second lower drum 228 by a shaft 232, fixing the second lower drum 228 for rotation by the second lower gear assembly 152. In at least one example embodiment, the second lower gear assembly 152 engages with the second upper gear assembly 150 to either drive or be driven by rotation of the second upper gear assembly 150. The second upper gear assembly 150 and the second lower gear assembly 152 determine the speed relationship between the second upper drum 146 and the second lower drum 228.

In at least one example embodiment, the second upper drum 146 and the second lower drum 228 are second pressing drums. In at least one example embodiment, the second upper drum 146 includes a smooth outer surface 158 and the second lower drum includes a smooth outer surface 236 for contacting the pressed sheet. However, in other example embodiments, the outer surfaces 158, 236 may each have a textured surface. In other example embodiments, the outer surfaces 158, 236 may each include a non-stick coating (not shown). The second pair of drums 144 may receive the pressed sheet and, as the second upper drum 146 and second lower drum 228 rotate, the second upper drum 146 presses the first pressed sheet against the second lower drum 228 to form the first pressed sheet into a second pressed sheet having a second thickness, as further described below.

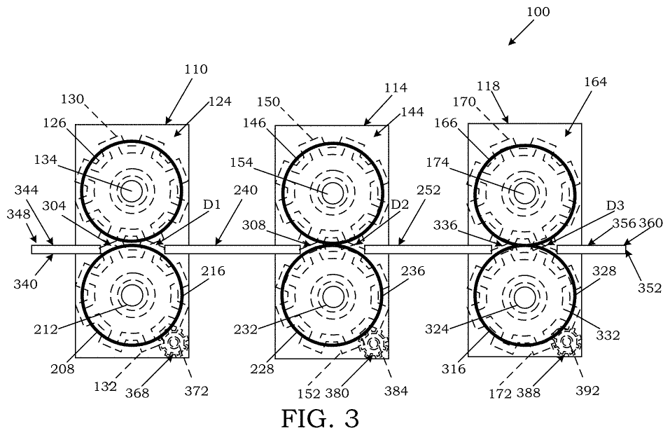

In at least one example embodiment, the third section 118 includes a third tower having a third housing 160, a third pair of gear assemblies 162, and a third pair of drums 164. The third pair of drums 164 includes a third upper drum 166 and a third lower drum 316 (further discussed below with reference to FIG. 3). The third housing 160 at least partially encloses the third upper drum 166 and the third lower drum 316. The third upper drum 166 is positioned adjacent to, and vertically above, the third lower drum 316. In at least one example embodiment, the third upper drum 166 and the third lower drum 316 are cylindrical drums. The third upper drum 166 and the third lower drum 316 may rotate within the third housing 160 to collectively process the second pressed sheet.

In at least one example embodiment, the third pair of gear assemblies 162 includes a third upper gear assembly 170 and a third lower gear assembly 172. The third upper drum 166 is rotated by the third upper gear assembly 170, which is outside of the third housing 160. In at least one example embodiment, the third upper gear assembly 170 includes one or more wheels having teeth. The teeth of each of the wheels intermesh to transfer movement from one wheel to another.

In at least one example embodiment, the third upper gear assembly 170 is connected to the third upper drum 166 by a shaft 174, fixing the third upper drum 166 for rotation by the third upper gear assembly 170. In at least one example embodiment, the third upper gear assembly 170 is rotated, either manually or by a motor, as described below, to rotate the third upper drum 166.

In at least one example embodiment, the third lower drum 316 is rotated by the third lower gear assembly 172, which is outside of the third housing 160 (see FIG. 3). In at least one example embodiment, the third lower gear assembly 172 includes one or more wheels having teeth. The teeth of each of the wheels intermesh to transfer movement from one wheel to another.

In at least one example embodiment, the third lower gear assembly 172 is connected to the third lower drum 316 by a shaft 324, fixing the third lower drum 316 for rotation by the third lower gear assembly 172. In at least one example embodiment, the third lower gear assembly 172 engages with the third upper gear assembly 170 to either drive or be driven by rotation of the third upper gear assembly 170. The third upper gear assembly 170 and the third lower gear assembly 172 determine the speed relationship between the third upper drum 166 and the third lower drum 316.

In at least one example embodiment, the third upper drum 166 and the third lower drum 316 are forming drums. The third pair of drums 164 receive the second pressed sheet and collectively form the second pressed sheet into a plurality of gum pieces, as further described below.

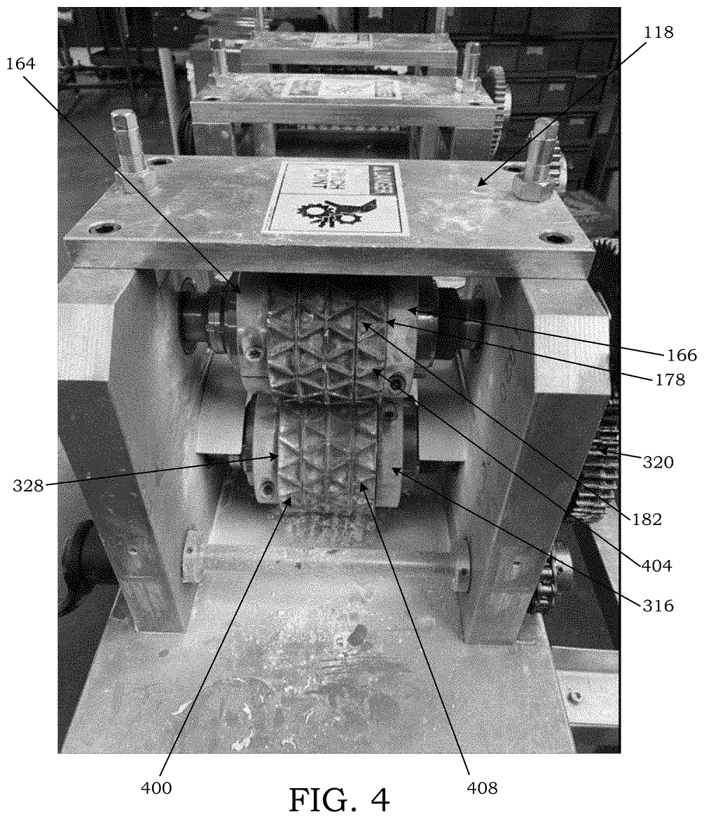

In at least one example embodiment, the third upper drum 166 includes a die 178 having a pattern of recessed portions 182 on an outer surface 184 thereof. In at least one example embodiment, the third lower drum 316 includes a die 328 having a pattern of recessed portions 400 on an outer surface 332 thereof (see FIG. 4). The die 328 is similar to the die 178. The pattern of recessed portions 182 and the pattern of recessed portions 400 may collectively form the pressed sheet into a scored sheet including the plurality of gum pieces.

In at least one example embodiment, the third pair of drums 164 receives the second pressed sheet, and the third upper drum 166 and third lower drum 316 rotate to form the second pressed sheet into a plurality of gum pieces, or a scored sheet including the plurality of gum pieces. In at least one example embodiment, the third pair of drums 164 cooperate to score the plurality of gum pieces in a scored sheet, as further described below. The scored sheet includes the plurality of gum pieces molded and attached in a single sheet. In at least one alternative example embodiment, the third pair of drums 164 cooperate to cut the plurality of gum pieces into individual, separate, pieces, as further described below.

In at least one example embodiment, a first plate 240 extends between the first section 110 and the second section 114, and, more specifically, between the first pair of drums 124 of the first section 110 and the second pair of drums 144 of the second section 114. In at least one example embodiment, the first plate 240 is positioned such that the first pressed sheet is deposited on the first plate 240 from the first pair of drums 124 in the first section 110, supported by the first plate 240, and fed into the second pair of drums 144 in the second section 114.

In at least one example embodiment, the first plate 240 includes a first surface 244 that contacts the first pressed sheet as the first pressed sheet moves through the apparatus 100. The first surface 244 is a substantially flat, planar surface extending along a top 248 of the first plate 240. In at least one example embodiment, the first surface 244 is a smooth surface to facilitate conveying the first pressed sheet from the first pair of drums 124 to the second pair of drums 144.

In at least one example embodiment, a second plate 252 extends between the second section 114 and the third section 118, and, more specifically, between the second pair of drums 144 of the second section 114 and the third pair of drums 164 of the third section 118. In at least one example embodiment, the second plate 252 is positioned such that the second pressed sheet is deposited on the second plate 252 from the second pair of drums 144 in the second section 114. The second pressed sheet is supported by the second plate 252, and fed into the third pair of drums 164 in the third section 118.

In at least one example embodiment, the second plate 252 includes a second surface 256 that contacts the second pressed sheet as the second pressed sheet moves through the apparatus 100. The second surface 256 is a flat, planar surface extending along a top 260 of the second plate 252. In at least one example embodiment, the second surface 256 is a smooth surface to facilitate conveying the second pressed sheet from the second pair of drums 144 to the third pair of drums 164.

In at least one example embodiment, the first section 110, second section 114, and third section 118 are supported on a frame 186. The frame 186 may include a planar support 190, such as a table or plate, which supports the first section 110, second section 114, and third section 118.

FIG. 3 is a schematic view of an apparatus for forming nicotine gum according to at least one example embodiment.

In at least one example embodiment, the apparatus 100 includes the first section 110 having the first pair of drums 124, the second section 114 having the second pair of drums 144, and the third section 118 having the third pair of drums 164. The first upper drum 126 and the first lower drum 208 of the first pair of drums 124 are vertically aligned, with the first upper drum 126 positioned above the first lower drum 208. The first upper drum 126 is spaced from the first lower drum 208 a first distance D1 to create a gap 304 therebetween. In at least one example embodiment, the distance D1 between the first upper drum 126 and first lower drum 208 ranges from about 4 millimeters (mm) to about 20 mm (e.g., about 8 mm to about 11 mm or about 9.5 mm to about 10 mm). Although the example range is provided, it is understood that the present disclosure is not limited thereto. The distance D1 may be dependent on the desired (or, alternatively, predetermined) nicotine gum product and may vary for different products, different target weights, or different desired (or, alternatively, predetermined) thicknesses.

In at least one example embodiment, the first upper drum 126 and first lower drum 208 are pressing drums that cooperate to collectively press the gum mixture to create a first pressed sheet. In at least one example embodiment, the first pressed sheet has a thickness equivalent to the distance D1 of the gap 304 between the first upper drum 126 and the first lower drum 208. Accordingly, in at least one example embodiment, the first pressed sheet has a thickness ranging from about 4 mm to about 20 mm (e.g., about 8 mm to about 11 mm or about 9.5 mm to about 10 mm).

In at least one example embodiment, the second upper drum 146 and the second lower drum 228 of the second pair of drums 144 are vertically aligned, with the second upper drum 146 positioned above the second lower drum 228. The second upper drum 146 is spaced from the second lower drum 228 a second distance D2 to create a gap 308 therebetween. In at least one example embodiment, the distance D2 between the second upper drum 146 and second lower drum 228 ranges from about 2 mm to about 18 mm (e.g., about 6 mm to about 9 mm or about 7.5 mm to about 8 mm). Although the example range is provided, it is understood that the present disclosure is not limited thereto. The distance D2 may be dependent on the desired (or, alternatively, predetermined) nicotine gum product and may vary for different products, different target weights, or different desired (or, alternatively, predetermined) thicknesses.

In at least one example embodiment, the second upper drum 146 and second lower drum 228 are pressing drums that cooperate to collectively further press the first pressed sheet to create a second pressed sheet. In at least one example embodiment, the second pressed sheet has a thickness equivalent to the distance D2 of the gap 308 between the second upper drum 146 and the second lower drum 228. Accordingly, in at least one example embodiment, the second pressed sheet has a thickness ranging from about 2 mm to about 18 mm (e.g., about 6 mm to about 9 mm, or about 7.5 mm to about 8 mm).

In at least one example embodiment, the third upper drum 166 and the third lower drum 316 are vertically aligned, with the third upper drum 166 positioned above the third lower drum 316. The third upper drum 166 is spaced from the third lower drum 316 a third distance D3 to create a gap 336 therebetween. In at least one example embodiment, the distance D3 between the third upper drum 166 and third lower drum 316 ranges from about 0 mm to about 0.2 mm (e.g., about 0.1 mm). Although an example range is provided, it is understood that the present disclosure is not limited thereto. The distance D3 may be dependent on the desired (or, alternatively, predetermined) nicotine gum product and may vary for different products. In at least one example embodiment, the distance D3 ranges from about 0.1 mm to about 0.2 mm to score the second pressed sheet into the scored sheet. In at least one alternative example embodiment, the distance D3 is about 0 mm, or the third upper drum 166 contacts the third lower drum 316, to cut the second pressed sheet into individual gum pieces.

In at least one example embodiment, a supply plate 340 extends upstream of the first section 110 and, more specifically, upstream of the first pair of drums 124 of the first section 110. In at least one example embodiment, the supply plate 340 is positioned such that the gum mixture is deposited on the supply plate 340, supported by the supply plate 340, and fed into the first pair of drums 124 in the first section 110.

In at least one example embodiment, the supply plate 340 includes a surface 344 that contacts the gum mixture. The surface 344 is a flat, planar surface extending along a top 348 of the supply plate 340. In at least one example embodiment, the surface 344 is a smooth surface to facilitate conveying the gum mixture to the first pair of drums 124.

In at least one example embodiment, a discharge plate 352 extends downstream of the third section 118 and, more specifically, downstream of the third pair of drums 164 of the third section 118. In at least one example embodiment, the discharge plate 352 is positioned such that the scored sheet or individual gum pieces are deposited on the discharge plate 352, supported by the discharge plate 352, and conveyed away from the third pair of drums 164 in the third section 118.

In at least one example embodiment, the discharge plate 352 includes a surface 356 that contacts the scored sheet or individual gum pieces. The surface 356 is a flat, planar surface extending along a top 360 of the discharge plate 352. In at least one example embodiment, the surface 356 is a smooth surface to facilitate conveying the scored sheet or individual gum pieces away from the third pair of drums 164.

In at least one example embodiment, during operation, the gum mixture is deposited on the supply plate 340. The first upper drum 126 and first lower drum 208 begin rotation. In at least one example embodiment, the first upper drum 126 rotates in a counterclockwise direction, while the first lower drum 208 rotates in a clockwise direction.

In at least one example embodiment, the first upper drum 126 rotates at a first speed and the first lower drum 208 rotates at a second speed. The first speed and the second speed are set such that there is consistent material transfer through the first upper drum 126 and first lower drum 208. In at least one example embodiment, the first speed is the same as the second speed and ranges from about 0 revolutions per minute (RPM) to about 20 RPM. In at least one alternative example embodiment, the first speed is different from the second speed. The first upper drum 126 may rotate at a speed ranging from about 0 RPM to about 20 RPM and the first lower drum 208 may rotate at a speed ranging from about 0 RPM to about 20 RPM. Having a different first speed and second speed may allow for imposed strain in the gum mixture to relax as the mixture passes through the apparatus 100. In at least one example embodiment, the first speed and the second speed are determined based on the speed that the plurality of gum pieces are removed from the third pair of drums 300 (for example, a speed that an operator removes the plurality of gum pieces from the third pair of drums 300). In at least one example embodiment, the speed that the plurality of gum pieces are removed from the third pair of drums 300 is related to the ingredients and proportions of ingredients of the gum material, the environmental conditions, the temperature, the coating effectiveness of the gum material, the geometry of the recessed portions 182, 400, or a combination of these.

In at least one example embodiment, the first upper drum 126 is rotated by the first upper gear assembly 130, and the first lower drum 208 is rotated by the first lower gear assembly 132. The first upper gear assembly 130 and the first lower gear assembly 132 determine the speed relationship between the first upper drum 126 and the first lower drum 208. In at least one example embodiment, a number of teeth on each gear, a size of each gear, and a number of gears for each of the first upper gear assembly 130 and the first lower gear assembly 132 determine the speed relationship.

In at least one example embodiment, the first upper gear assembly 130, the first lower gear assembly 132, or a combination thereof, are manually rotated. A crank 368 includes a crank gear 372 that engages with the first lower gear assembly 132 to drive rotation of the first lower gear assembly 132. Rotation of the first lower gear assembly 132 rotates the first lower drum 208 and drives rotation of the first upper gear assembly 130. Rotation of the first upper gear assembly 130 rotates the first upper drum 126. In at least one alternative embodiment, the crank 368 engages the first upper gear assembly 130, having a reverse effect.

In at least one example embodiment, the gum mixture is fed into the gap 304 separating the first upper drum 126 from the first lower drum 208. Rotation of the first upper drum 126 and first lower drum 208 draws the gum mixture through the gap 304. The gum mixture is pressed between the first upper drum 126 and first lower drum 208 into the first pressed sheet. As previously stated, in at least one example embodiment, the first pressed sheet includes a thickness ranging from about 4 mm to about 20 mm (e.g., about 8 mm to about 11 mm or about 9.5 mm to about 10 mm). The first pressed sheet exits the gap 304 and first pair of drums 124 onto the first plate 240.

In at least one example embodiment, the first pressed sheet is transferred along the first plate 240 from the first pair of drums 124 to second the pair of drums 144. The second upper drum 146 and second lower drum 228 of the second pair of drums 144 begins rotation. In at least one example embodiment, the second upper drum 146 rotates in a counterclockwise direction, while the second lower drum 228 rotates in a clockwise direction.

In at least one example embodiment, the first pair of drums 124 may be spaced from the second pair of drums 144 by a distance ranging from about 100 mm to about 1500 mm (e.g., from about 200 mm to about 400 mm, or about 305 mm). In at least one example embodiment, the first pressed sheet may fully exit the first pair of drums 124 before being fed into the second pair of drums 144. In at least one alternative embodiment, the first pressed sheet may be pressed within the first pair of drums 124 on a first portion and pressed within the second pair of drums 144 on a second portion simultaneously. Thus, the material of the first pressed sheet may extend between multiple pairs of drums of the first pair of drums 124, the second pair of drums 144, and the third pair of drums 164.

In at least one example embodiment, the second upper drum 146 rotates at a first speed and the second lower drum 228 rotates at a second speed. The first speed and the second speed are set such that there is consistent material transfer through the second upper drum 146 and second lower drum 228. In at least one example embodiment, the first speed is the same as the second speed and ranges from about 0 RPM to about 20 RPM. In at least one alternative example embodiment, the first speed is different from the second speed. The speed of the second upper drum 146 may range from about 0 RPM to about 20 RPM, and the speed of the second lower drum 228 may range from about 0 RPM to about 20 RPM. Having a different first speed and second speed may allow for imposed strain in the material of the first pressed sheet to relax as the first pressed sheet passes through the apparatus 100. In at least one example embodiment, the first speed and the second speed are determined based on the speed that the plurality of gum pieces are removed from the third pair of drums 300 (for example, a speed that an operator removes the plurality of gum pieces from the third pair of drums 300). In at least one example embodiment, the speed that the plurality of gum pieces are removed from the third pair of drums 300 is related to the ingredients and proportions of ingredients of the gum material, the environmental conditions, the temperature, the coating effectiveness of the gum material, the geometry of the recessed portions 182, 400, or a combination of these.

In at least one example embodiment, the first speed of the second upper drum 146 and the second speed of the second lower drum 228 are the same as the first speed of the first upper drum 126 and the second speed of the first lower drum 208, respectively. In at least one alternative example embodiment, the first speed of the second upper drum 146 and the second speed of the second lower drum 228 are different from the first speed of the first upper drum 126 and the second speed of the first lower drum 208.

In at least one example embodiment, the second upper drum 146 is rotated by the second upper gear assembly 150, and the second lower drum 228 is rotated by the second lower gear assembly 152. The second upper gear assembly 150 and the second lower gear assembly 152 determine the speed relationship between the second upper drum 146 and the second lower drum 228. In at least one example embodiment, a number of teeth on each gear, a size of each gear, and a number of gears for each of the second upper gear assembly 150 and the second lower gear assembly 152 determine the speed relationship.

In at least one example embodiment, the second upper gear assembly 150, the second lower gear assembly 152, or a combination thereof, are manually rotated. A crank 380 includes a crank gear assembly 384 that engages with the second lower gear assembly 152 to drive rotation of the second lower gear assembly 152. Rotation of the second lower gear assembly 152 rotates the second lower drum 228 and drives rotation of the second upper gear assembly 150. Rotation of the second upper gear assembly 150 rotates the second upper drum 146. In at least one alternative embodiment, the crank 380 may engage the second upper gear assembly 150, having a reverse effect.

In at least one example alternative embodiment, the second upper gear assembly 150, the second lower gear assembly 152, or a combination thereof, is engaged with the first upper gear assembly 130, the first lower gear assembly 132, or a combination thereof, of the first section 110 and drives, or is driven by, the first upper gear assembly 130, the first lower gear assembly 132, or a combination thereof.

In at least one example embodiment, the first pressed sheet is fed into the gap 308 separating the second upper drum 146 from the second lower drum 228. Rotation of the second upper drum 146 and second lower drum 228 draws the first pressed sheet through the gap 308. The first pressed sheet is further pressed between the second upper drum 146 and second lower drum 228 into the second pressed sheet. As previously stated, in at least one example embodiment, the second pressed sheet has a thickness ranging from about 2 mm to about 18 mm (e.g., about 6 mm to about 9 mm or about 7.5 mm to about 8 mm). The second pressed sheet exits the gap 308 and second pair of drums 142 onto the second plate 252.

In at least one example embodiment, the second pressed sheet is transferred along the second plate 252 from the second pair of drums 144 to the third pair of drums 164. The third upper drum 166 and third lower drum 316 of the third pair of drums 164 begin rotation. In at least one example embodiment, the third upper drum 166 rotates in a counterclockwise direction, while the third lower drum 316 rotates in a clockwise direction.

In at least one example embodiment, the third upper drum 166 rotates at a first speed and the third lower drum 316 rotates at a second speed. The first speed and the second speed are set such that there is consistent material transfer through the third upper drum 166 and third lower drum 316. In at least one example embodiment, the first speed is the same as the second speed and ranges from about 0 RPM to about 20 RPM. In at least one alternative example embodiment, the first speed is different from the second speed. The speed of the third upper drum 166 may range from about 0 RPM to about 20 RPM and the speed of the third lower drum 316 may range from about 0 RPM to about 20 RPM. Having a different first speed and second speed may allow for imposed strain in the material of the second pressed sheet to relax as the second pressed sheet passes through the apparatus 100. In at least one example embodiment, the first speed and the second speed are determined based on the speed that the plurality of gum pieces are removed from the third pair of drums 300 (for example, a speed that an operator removes the plurality of gum pieces from the third pair of drums 300). In at least one example embodiment, the speed that the plurality of gum pieces are removed from the third pair of drums 300 is related to the ingredients and proportions of ingredients of the gum material, the environmental conditions, the temperature, the coating effectiveness of the gum material, the geometry of the recessed portions 182, 400, or a combination of these.

In at least one example embodiment, the first speed of the third upper drum 166 and the second speed of the third lower drum 316 are the same as the first speed of the first upper drum 126 and the second speed of the first lower drum 208, respectively. In at least one example embodiment, the first speed of the third upper drum 166 and the second speed of the third lower drum 316 are the same as the first speed of the second upper drum 146 and the second speed of the second lower drum 228, respectively. In at least one alternative example embodiment, the first speed of the third upper drum 166 and the second speed of the third lower drum 316 are different from the first speed of the first upper drum 126, the second speed of the first lower drum 208, the first speed of the second upper drum 146, and the second speed of the second lower drum 228.

In at least one example embodiment, the third upper drum 166 is rotated by the third upper gear assembly 170, and the third lower drum 316 is rotated by the third lower gear assembly 172. The third upper gear assembly 170 and the third lower gear assembly 172 determine the speed relationship between the third upper drum 166 and the third lower drum 316. In at least one example embodiment, a number of teeth on each gear, a size of each gear, and a number of gears for each of the third upper gear assembly 170 and the third lower gear assembly 172 determine the speed relationship.

In at least one example embodiment, the third upper gear assembly 170, the third lower gear assembly 172, or a combination thereof, are manually rotated. A crank 388 includes a crank gear 392 that engages with the third lower gear assembly 172 to drive rotation of the third lower gear assembly 172. Rotation of the third lower gear assembly 172 rotates the third lower drum 316 and drive rotation of the third upper gear assembly 170. Rotation of the third upper gear assembly 170 rotates the third upper drum 166. In at least one alternative embodiment, the crank 388 engages the third upper gear assembly 170, having a reverse effect.

In at least one example alternative embodiment, the third upper gear assembly 170, the third lower gear assembly 172, or a combination thereof, is engaged with the first upper gear assembly 130, the first lower gear assembly 132, the second upper gear assembly 150, the second lower gear assembly 152, or a combination thereof, and drives, or is driven by, the first upper gear assembly 130, the first lower gear assembly 132, the second upper gear assembly 150, the second lower gear assembly 152, or a combination thereof. In at least one example embodiment, the second pressed sheet is fed into the gap 336 separating the third upper drum 166 from the third lower drum 316. Rotation of the third upper drum 166 and third lower drum 316 draws the second pressed sheet through the gap 336. The second pressed sheet is formed between the third upper drum 166 and third lower drum 316. In at least one example embodiment, the second pressed sheet is scored by the third upper drum 166 and the third lower drum 316, forming a scored sheet. The scored sheet defines the plurality of gum pieces. In at least one example embodiment, the plurality of gum pieces remain connected in the scored sheet. In at least one alternative example embodiment, the second pressed sheet is cut by the third upper drum 166 and the third lower drum 316 into the individual, separate, plurality of gum pieces. The plurality of gum pieces exits the gap 336 and third pair of drums 164 onto the discharge plate 352.

In at least one example embodiment, the plurality of gum pieces is transferred along the discharge plate 352 away from the third pair of drums 164.

FIG. 4 is an end perspective view of an apparatus for forming nicotine gum according to at least one example embodiment.

In at least one example embodiment, the die 178 on the third upper drum 166 includes a pattern of recessed portions 182 and non-recessed portions 404. The die 328 on the third lower drum 316 also includes a pattern of recessed portions 400 and non-recessed portions 408. The recessed portions 400 correspond to the recessed portions 182. The non-recessed portions 408 correspond to the non-recessed portions 404. As the third upper drum 166 and third lower drum 316 rotate, the recessed portions 182 align with the recessed portions 400 and the non-recessed portions 404 align with the non-recessed portions 408.

In at least one example embodiment, the non-recessed portions 404 define the boundaries of the recessed portions 182 and the non-recessed portions 408 define the boundaries of the recessed portions 400. As the third upper drum 166 and third lower drum 316 rotate, the non-recessed portions 404 align with the non-recessed portions 408 to score or cut the second pressed sheet and define the plurality of gum pieces.

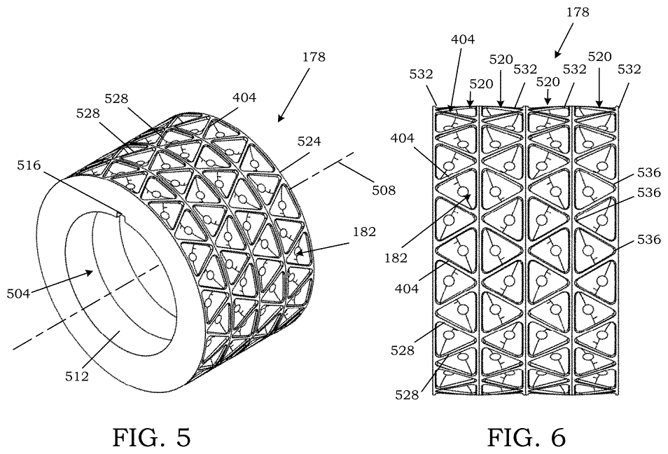

FIG. 5 is a perspective view of a die used in an apparatus for forming nicotine gum according to at least one example embodiment.

In at least one example embodiment, the die 178 is a cylindrical or tubular die having an aperture 504 extending along a longitudinal axis 508 of the die 500. The aperture 504 is defined by an inner wall 512 of the die 500.

In at least one example embodiment, the die 178 is formed of a polymer, such as a plastic, a metal, such as aluminum or stainless steel, or any other suitable material. In at least one example embodiment, the die 178 is formed by three-dimensional (3-D) printing, stamping, bending, or any other suitable process.

In at least one example embodiment, the die 178 includes a width corresponding to a width of the third upper drum 164 which ranges from about 5 mm to about 480 mm (e.g., from about 44 mm to about 175 mm, or from about 88 mm to about 132 mm). In at least one alternative example embodiment, the width of the die 178 is less than the width of the third upper drum 164. The die 178 additionally includes an inner diameter d1 corresponding to a diameter of the third upper drum and ranging from about 100 mm to about 300 mm or larger (e.g., from about 130 mm to about 140 mm) and an outer diameter d2 ranging from about 100 mm to about 400 mm or larger (e.g., from about 135 mm to about 145 mm).

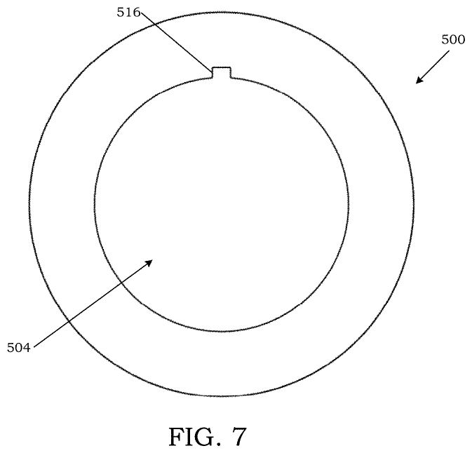

In at least one example embodiment, the die 178 is configured to be disposed on the third upper drum 166 or the third lower drum 316 in the third section 118. In at least one example embodiment the die 178 includes a channel 516 in the inner wall 512 of the die 178 (see FIG. 7). The channel 516 extends parallel to the longitudinal axis 508 along at least a portion of a length of the die 178. The channel 516 is configured to align the die 178 on the third upper drum 166 or the third lower drum 316. In at least one example embodiment, the channel 516 engages with a rail (not shown) on a surface of the third upper drum 166 or the third lower drum 316 to align the die 178 on the third upper drum 166 or the third lower drum 316.

In at least one example embodiment, the die 178 includes the plurality of recessed portions 182 and the plurality of non-recessed portions 404. In at least one example embodiment, the plurality of non-recessed portions 404 define a boundary of each of the plurality of recessed portions 182. In at least one example embodiment, the plurality of non-recessed portions includes a plurality of segments 528 connected together. Each segment 528 of the plurality of segments 528 defines a boundary of two different recessed portions 182, to reduce wasted material during the scoring or cutting of the plurality of gum pieces.

In at least one example embodiment, the die 328 is the same as the die 178 except that the die 328 is positioned on the third lower drum 316.

FIG. 6 is a front view of a die used in an apparatus for forming nicotine gum according to at least one example embodiment.

In at least one example embodiment, the die 500 is formed of a plurality of rings 532. In at least one example embodiment, each ring 532 may have a width ranging from about 5 mm to about 40 mm (e.g., from about 15 mm to about 30 mm, or about 22 mm). The die 500 may include the plurality of rings 532 ranging from about 1 ring to about 12 rings (e.g., from about 2 rings to about 6 rings, or about 4 rings).

Each ring 532 includes a single row of recessed portions 520. In at least one example embodiment, the recessed portions 520 are arranged in an alternating arrangement to more efficiently utilize space. As shown in FIG. 6, each of the recessed portions 520 may include a triangular cross-sectional shape along an upper surface of the non-recessed portions 404 adjacent the recessed portion 520. In at least one example embodiment, each of the recessed portions 520 includes a vertex 536 in an opposite direction from the vertex 536 of the adjacent recessed portions 520, such that the recessed portions alternate pointing directions along the row of recessed portions 520 for each ring 532. Additionally, the rings 532 are positioned such that the vertex 526 for adjacent recessed portions 520 on different rings 532 alternate pointing directions. The vertex 526 of the recessed portion 520 on one ring 532, therefore, is either positioned adjacent or opposite the vertex 526 of the recessed portion 420 on another ring 532.

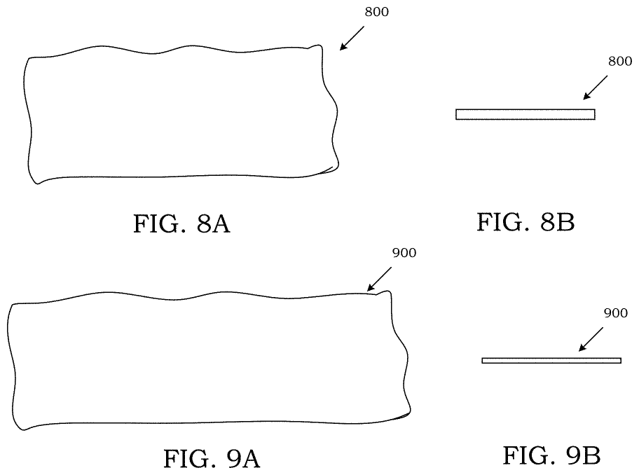

In at least one example embodiment, the plurality of recessed portions 520 are shaped to manufacture one of a variety of different sizes and shapes (as described in greater detail below and shown in FIGS. 8A-8F). In at least one example embodiment, the plurality of recessed portions 520 are shaped to form a half of each of the plurality of gum pieces (for example, the upper half or lower half), such that the die on the third upper drum 166 cooperates with the die on the third lower drum 316 to form a whole of each of the plurality of gum pieces (i.e., the upper half and the lower half).

In at least one example embodiment, each of the plurality of recessed portions 520 has a size and/or shape that forms a half (for example, the upper half or lower half) of the nicotine gum piece. Accordingly, a complimentary pair of recessed portions 520, which are aligned on opposing sides of the second pressed sheet during operation, define the size and shape of a nicotine gum piece. The size and shape of the nicotine gum piece promotes desired (or, alternatively, predetermined) positioning of the nicotine gum piece within an oral cavity and/or a package. In at least one example embodiment, each of the plurality of recessed portions 520 has dimensions ranging from about 1 mm to about 25 mm (e.g., about 1 mm to about 10 mm, about 1 mm to about 5 mm, about 5 mm to about 25 mm, about 5 mm to about 10 mm, about 10 mm to about 15 mm, about 15 mm to about 20 mm, or about 20 mm to about 25 mm). In at least one example embodiment, each of the plurality of recessed portions 520 has a first dimension (e.g., smallest dimension or thickness) ranging from about 0.5 mm to about 5 mm (e.g., about 2.5 mm). In at least one example embodiment, each of the plurality of recessed portions 520 has a largest dimension (e.g., diameter, height, or width) ranging from about 5 mm to about 25 mm (e.g., about 12 mm). Each of the plurality of recessed portions 520 forms a nicotine gum piece having a weight ranging from about 1 g to about 10 g (e.g., about 1 g to about 5 g, about 2 g to about 4 g, or about 5 g to about 10 g).

In at least one example embodiment, each nicotine gum piece defines a thickness and a cross-sectional shape perpendicular to a thickness. The thickness and cross-section shape of the nicotine gum piece correspond to a thickness and cross-sectional shape of one complimentary pair of the plurality of recessed portions 520. As used herein, “thickness” refers to the smallest dimension of a nicotine gum piece. In at least one example embodiment, a nicotine gum piece has a thickness that is largest in a center and tapers toward each of the vertices. In at least one alternative example embodiment, a nicotine gum piece has a substantially uniform thickness. While not shown, each of the plurality of recessed portions 520 described herein may include rounded edges to form the plurality of gum pieces having rounded edges.

FIG. 7 is a side view of a die used in an apparatus for forming nicotine gum according to at least one example embodiment.

In at least one example embodiment, the plurality of rings 532 each include the channel 516. The channel 516 aligns the plurality of rings 532 relative to one another for assembly on the third upper drum 166 or the third lower drum 316.

In at least one example embodiment, the plurality of rings 532 are not attached. In at least one alternative example embodiment, the plurality of rings are fixed together by adhesive, a fastener, or another fixing method. In at least one alternative example embodiment, the plurality of rings 532 are formed as a single, monolithic part. In at least one alternative example embodiment, the plurality of rings 532 are not fixed together.

In at least one example embodiment, the plurality of rings 532 are press-fit onto the third upper drum 166 or the third lower drum 316. In at least one alternative example embodiment, the plurality of rings 532 are otherwise secured on the third upper drum 166 or the third lower drum 316. In at least one example embodiment, the plurality of rings 532 are secured on the third upper drum 166 or the third lower drum 316 by a pair of tightening nuts on opposing sides of the respective third upper drum 166 or third lower drum 316.

FIG. 8A is a top view of a first pressed sheet according to at least one example embodiment.

In at least one example embodiment, a first pressed sheet 800 is pressed by the first pair of drums 124 (as shown in FIG. 2). In at least one example embodiment, the gum mixture is fed into the gap 304, pressed by the first pair of drums 124, and exits the gap 304 as the first pressed sheet 800.

FIG. 8B is a side view of a first pressed sheet according to at least one example embodiment.

In at least one example embodiment, the first pressed sheet 800 includes a thickness ranging from about 4 mm to about 20 mm (e.g., about 8 mm to about 11 mm or about 9.5 mm to about 10 mm). The thickness of the first pressed sheet 800 may be chosen to prepare the first pressed sheet to be fed into the second pair of drums 144. Providing the first pressed sheet at a thickness near (for example, ranging within about 0.5 mm to about 5 mm, or about 1 mm to about 3 mm, or about 2 mm) the target thickness of the second pressed sheet assists the second pair of drums 144 in providing a uniform target thickness sheet to the third pair of drums 300.

FIG. 9A is a top view of a second pressed sheet according to at least one example embodiment.

In at least one example embodiment, a second pressed sheet 900 is pressed by the second pair of drums 144. In at least one example embodiment, the first pressed sheet 800 is fed into the gap 308 (shown in FIG. 3), pressed by the second pair of drums 144, and exits the gap 308 as the second pressed sheet 900.

FIG. 9B is a side view of a second pressed sheet according to at least one example embodiment.

In at least one example embodiment, the second pressed sheet 900 has a thickness ranging from about 2 mm to about 18 mm (e.g., about 4 mm to about 16 mm, about 6 mm to about 14 mm, about 8 mm to about 12 mm, about 9 mm to about 11 mm, about 6 mm to about 9 mm, or about 7.5 mm to about 8 mm). The thickness of the second pressed sheet 900 may be chosen based on a thickness needed for the gum material to fill the recessed portions of the die, the gum material necessary to achieve a target weight per gum piece, or a combination of these.

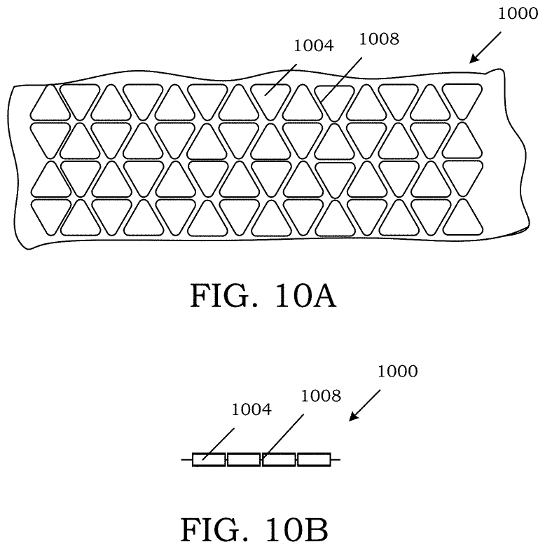

FIG. 10A is a top view of a scored sheet according to at least one example embodiment.

In at least one example embodiment, a scored sheet 1000 is formed by the third pair of drums 164. In at least one example embodiment, the second pressed sheet 900 is fed into the gap 336 (shown in FIG. 3), scored by the third pair of drums 164, and exits the gap 336 as the scored sheet 1000.

In at least one example embodiment, the scored sheet 1000 includes the plurality of gum pieces 1004 and connectors 1008 between the plurality of gum pieces 1004, such that the plurality of gum pieces 1004 exit the apparatus 100 as the scored sheet 1000. The plurality of gum pieces 1004 and the connectors 1008 are a single monolithic piece formed from the second pressed sheet 900. Each of the plurality of gum pieces 1004 takes a corresponding shape of one recessed portion of the plurality of recessed portions 182, 400. Following formation of the scored sheet 1000, the plurality of gum pieces 1004 may be separated to form a plurality of individual, separated gum pieces.

In at least one example embodiment, the plurality of gum pieces 1004 may be separated manually or automatically. Manual separation may be performed by a machine operator. During manual separation, the plurality of gum pieces 1004 may be pulled apart, breaking the connectors 1008. Automatic separation may be performed by a portion of the apparatus 100 and may include shaking, cutting, or other separating functions.

FIG. 10B is a side view of a scored sheet according to at least one example embodiment.

In at least one example embodiment, the scored sheet 1000 has a first thickness for each of the plurality of gum pieces 1004 and a second thickness for each of the connectors 1008. The first thickness is defined by a thickness or depth of each of the plurality of recessed portions 182, 400 (times two, for top and bottom) and ranges from about 4 mm to about 36 mm (e.g., about 6 mm to about 34 mm, about 8 mm to about 32 mm, about 10 mm to about 30 mm, about 12 mm to about 28 mm, about 14 mm to about 26 mm, about 16 mm to about 24 mm, about 18 mm to about 22 mm, about 12 mm to about 18 mm or about 15 mm to about 16 mm). The second thickness ranges from about 0.1 mm to about 0.2 mm.

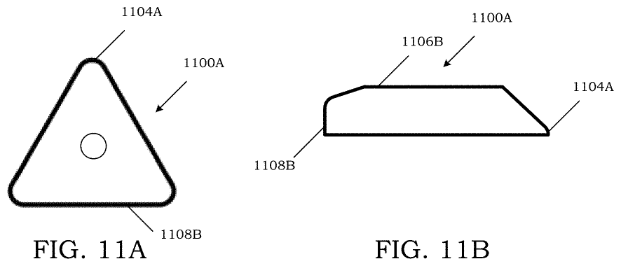

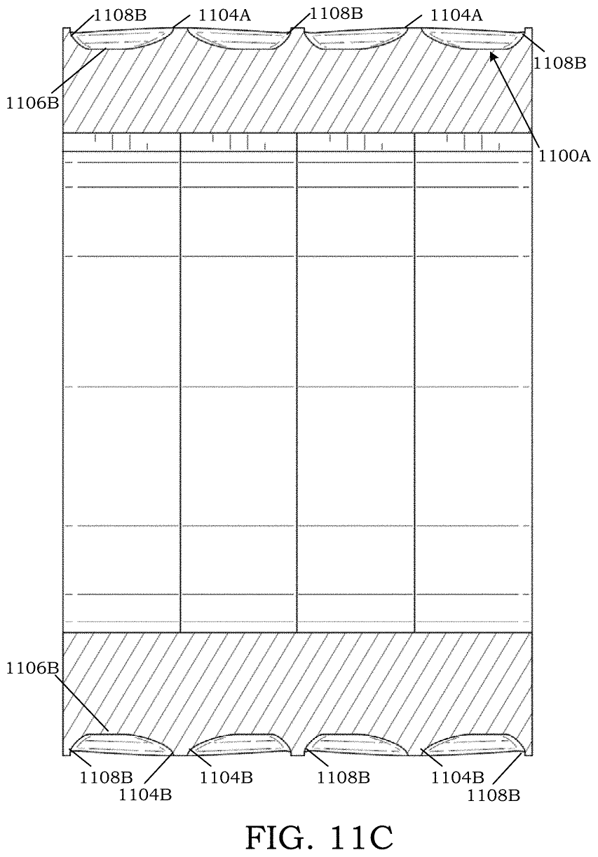

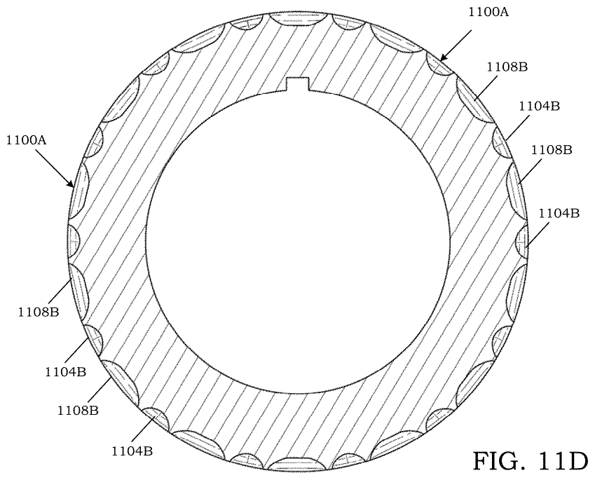

FIG. 11A is a cross-sectional view of a recessed portion of a die for forming gum pieces having a triangular cross-section along an upper surface of a non-recessed portion according to at least one example embodiment.

In at least one example embodiment, a recessed portion 1100A of the plurality of recessed portions 520 has a substantially triangular cross-section. The cross-section of the recessed portion 1100A has reflection symmetry about a center plane. In at least one example embodiment, the recessed portion 1100A includes three rounded corners, or vertices 1104A. In other example embodiments, the corners may not be rounded.

In at least one example embodiment, the recessed portion 1100A forms a portion of a nicotine gum piece (for example, an upper half or a lower half) having a shield-shape (e.g., the nicotine gum piece has a substantially triangular cross-section perpendicular a thickness). The nicotine gum piece has symmetry about a center plane. In at least one example embodiment, the nicotine gum piece formed by the recessed portion 1100A includes three rounded corners. In other example embodiments, the corners may not be rounded.

FIG. 11B is a cross-sectional view of a recessed portion of a die for forming gum pieces having a cross-section through a thickness and bisecting a vertex according to at least one example embodiment.

In at least one example embodiment, the cross-section through the thickness of the recessed portion 1100A tapers from a plateau 1106B to a rounded point at the vertex 1104A. In at least one alternative example embodiment, the point is not a rounded point at the vertex 1104A.

In at least one example embodiment, the cross-section through the thickness of the recessed portion 1100A slightly tapers from the plateau 1106B and squares-off on a side 1108B of the cross-section opposite the vertex 1104A. The cross-section includes rounded corners where the taper transitions to the squared side 1108B.

In at least one alternative example embodiment, the cross-section through the thickness of the recessed portion 1100A is half of an oval-shape. The cross-section through the thickness tapers from the plateau 1106B to parabola shape, or U-shape, at the vertex 1104A and tapers from the plateau 1106B to a parabola shape, or U-shape, on the side 1108B opposite the vertex 1104A. In at least one example embodiment, a radius of the parabola at the vertex 1104A is about the same as a radius of the parabola on the side 1108B opposite the vertex 1104A. In at least one alternative example embodiment, the radius of the parabola at the vertex 1104A is less than the radius of the parabola on the side 1108B.

In at least one alternative example embodiment, the cross-section through the thickness includes a parabola shape, or U-shape, at the vertex 1104A and a parabola shape, or U-shape, on the side 1108B opposite the vertex 1104A. The cross-section extends straight across and does not taper in thickness, such that the cross-section forms half of a capsule shape. In at least one example embodiment, a radius of the parabola at the vertex 1104A is about the same as a radius of the parabola on the side 1108B opposite the vertex 1104A.

In at least one alternative example embodiment, the cross-section through the thickness of the recessed portion 1100A slightly tapers from the plateau 1106B and squares-off at the vertex 1104A and the side 1108B opposite the vertex 1104A. In at least one example embodiment, the side 1108B has a thickness greater than a thickness of the vertex 1104A. In at least one alternative example embodiment, the thickness of the side 1108B is about the same as the thickness of the vertex 1104A.

In at least one alternative example embodiment, the cross-section through the thickness of the recessed portion tapers from the plateau 1106B to a rounded point at the vertex 1104A and tapers from the plateau 1106B to a rounded point on the side 1108B opposite the vertex 1104A. In at least one example embodiment, a radius of the rounded point at the vertex 1104A is the same as a radius of the rounded point on the side 1108B opposite the vertex 1104A. In at least one alternative example embodiment, the radius of the rounded point at the vertex 1104A is less than the radius of the rounded point on the side 1108B.

In at least one alternative example embodiment, the cross-section through the thickness of the recessed portion tapers from a rounded point at a midpoint between the vertex 1104A and the side 1108B opposite the vertex 1104A.

FIG. 11C is a cross-sectional view of a die cut along a longitudinal axis of the die and having recessed portions bisected through a vertex thereof according to at least one example embodiment.

In at least one example embodiment, the cross-section through the thickness of each of the recessed portions 1100A tapers from the plateau 1106B to the rounded point at the vertex 1104A. The cross-section through the thickness of the recessed portion 1100A tapers from the plateau 1106B and squares-off on the side 1108B of the cross-section opposite the vertex 1104A. The cross-section includes rounded corners where the taper transitions to the squared side 1108B.

FIG. 11D is a cross-sectional view of a die cut along an axis orthogonal to a longitudinal axis of the die and having recessed portions according to at least one example embodiment.

In at least one example embodiment, the vertex 1104A is rounded while the side 1108B opposite the vertex 1104A is elongated. Each of the three vertices 1104A are rounded points.

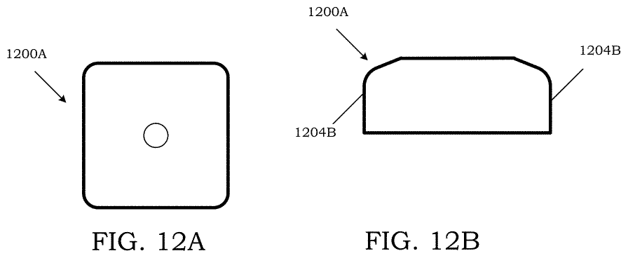

FIG. 12A is a cross-sectional view of a recessed portion of a die along an upper surface of a non-recessed portion for forming gum pieces having a square cross-section according to at least one example embodiment.

In at least one example embodiment, a recessed portion 1200A of the plurality of recessed portions 520 has a substantially square-shaped cross-section. Recessed portion 1200A is the same as recessed portion 1100A, except that recessed portion 1200A includes a substantially square cross-section. The cross-section of the recessed portion 1200A has reflection symmetry about a center plane. In at least one example embodiment, the recessed portion 1200A includes four rounded corners. In other example embodiments, the corners may not be rounded.

In at least one example embodiment, the recessed portion 1200A forms a portion of a nicotine gum piece (for example, an upper half or a lower half) that is substantially cube-shaped (e.g., the nicotine gum piece has a substantially square cross-section perpendicular to a thickness). In at least one example embodiment, the square cross-section has rounded corners. In other example embodiments, the corners may not be rounded.

FIG. 12B is a cross-sectional view of a recessed portion of a die for forming gum pieces having a cross-section through a thickness according to at least one example embodiment.

In at least one example embodiment, the cross-section through the thickness of the recessed portion 1200A slightly tapers and squares-off on a side 1204B of the cross-section, forming half of a substantially cube-shaped nicotine gum piece. In at least one alternative example embodiment, the recessed portion 1200 does not taper and includes rounded corners or pointed corners.

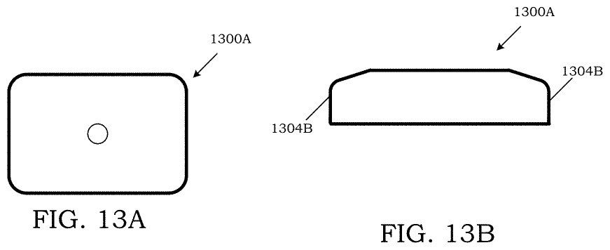

FIG. 13A is a cross-sectional view of a recessed portion of a die along an upper surface of a non-recessed portion for forming gum pieces having a rectangular cross-section according to at least one example embodiment.

In at least one example embodiment, a recessed portion 1300A of the plurality of recessed portions 520 has a substantially rectangular-shaped cross-section. Recessed portion 1300A is the same as recessed portion 1100A, except that recessed portion 1300A includes a substantially rectangular cross-section. The cross-section of the recessed portion 1300A has reflection symmetry about a center plane. In at least one example embodiment, the recessed portion 1300A includes four rounded corners. In other example embodiments, the corners may not be rounded.

In at least one example embodiment, the recessed portion 1300A forms a portion of a nicotine gum piece (for example, an upper half or a lower half) that is substantially rectangular-prism-shaped (e.g., the nicotine gum piece has a substantially rectangular cross-section perpendicular to a thickness). In at least one example embodiment, the rectangular cross-section has rounded corners. In other example embodiments, the corners may not be rounded.

FIG. 13B is a cross-sectional view of a recessed portion of a die for forming gum pieces having a cross-section through a thickness according to at least one example embodiment.

In at least one example embodiment, the cross-section through the thickness of the recessed portion 1300A slightly tapers and squares-off on a side 1304B of the cross-section, forming half of the rectangular-prism-shaped nicotine gum piece.

In at least one alternative example embodiment, the cross-section tapers to a rounded point (similar to the point 1104A in FIG. 11B) on each side 1304B of the cross-section. In at least one alternative example embodiment, the point is not a rounded point.

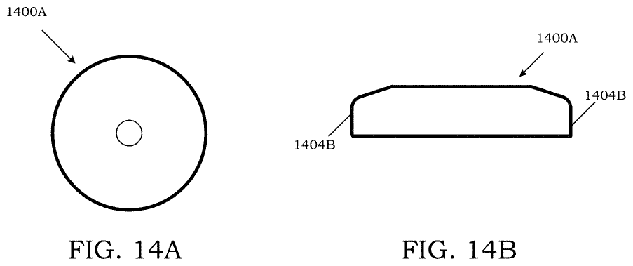

FIG. 14A is a cross-sectional view of a recessed portion of a die along an upper surface of a non-recessed portion for forming gum pieces having a circular cross-section according to at least one example embodiment.

In at least one example embodiment, a recessed portion 1400A of the plurality of recessed portions 520 has a substantially circular cross-section. Recessed portion 1400A is the same as recessed portion 1100A, except that recessed portion 1400A includes a substantially circular cross-section. The cross-section of the recessed portion 1400A has reflection symmetry about a center plane.

In at least one example embodiment, the recessed portion 1400A forms a portion of a nicotine gum piece (for example, an upper half or a lower half) that is substantially disk-shaped (e.g., the nicotine gum piece has a substantially circular cross-section perpendicular to a thickness).

FIG. 14B is a cross-sectional view of a recessed portion of a die for forming gum pieces having a cross-section through a thickness according to at least one example embodiment.

In at least one example embodiment, the cross-section through the thickness of the recessed portion 1300A slightly tapers and squares-off on a side 1404B of the cross-section, forming half of the disc-shaped nicotine gum piece.

In at least one alternative example embodiment, the cross-section tapers to a rounded point (similar to the point 1104A in FIG. 11B) on each side 1404B of the cross-section. In at least one alternative example embodiment, the point is not a rounded point.

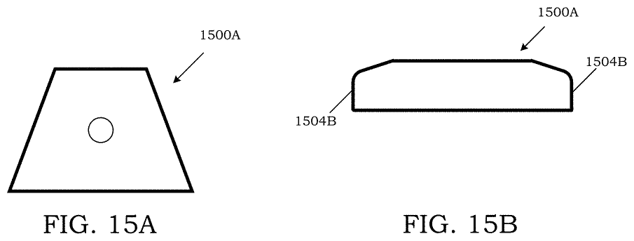

FIG. 15A is a cross-sectional view of a recessed portion of a die along an upper surface of a non-recessed portion for forming gum pieces having a trapezoidal cross-section according to at least one example embodiment.

In at least one example embodiment, a recessed portion 1500A of the plurality of recessed portions 520 has a substantially trapezoidal cross-section. Recessed portion 1500A is the same as recessed portion 1100A, except that recessed portion 1500A includes a substantially trapezoidal cross-section. The cross-section of the recessed portion 1500A has reflection symmetry about a center plane. In at least one example embodiment, the recessed portion 1500A includes four rounded corners. In other example embodiments, the corners may not be rounded.

FIG. 15B is a cross-sectional view of a recessed portion of a die for forming gum pieces having a cross-section through a thickness according to at least one example embodiment.

In at least one example embodiment, the cross-section through the thickness of the recessed portion 1500A slightly tapers and squares-off on a side 1504B of the cross-section, forming half (for example, an upper half or a lower half) of a trapezoidal-prism-shaped nicotine gum piece.

In at least one alternative example embodiment, the cross-section tapers to a rounded point (similar to the point 1104A in FIG. 11B) on each side 1504B of the cross-section. In at least one alternative example embodiment, the point is not a rounded point.

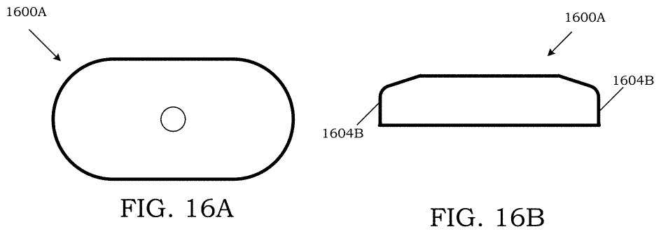

FIG. 16A is a cross-sectional view of a recessed portion of a die along an upper surface of a non-recessed portion for forming gum pieces having a stadium or oblong cross-section according to at least one example embodiment.

In at least one example embodiment, a recessed portion 1600A of the plurality of recessed portions 520 has a substantially stadium-shaped, or oval, cross-section. Recessed portion 1600A is the same as recessed portion 1100A, except that recessed portion 1600A includes a substantially stadium-shaped cross-section. The cross-section of the recessed portion 1600A has reflection symmetry about a center plane.

In at least one example embodiment, the recessed portion 1600A forms a portion of a nicotine gum piece (for example, an upper half or a lower half) that is substantially capsule-shaped (e.g., the nicotine gum piece has a substantially stadium-shaped, or oval-shaped, cross-section perpendicular to a thickness).

FIG. 16B is a cross-sectional view of a recessed portion of a die for forming gum pieces having a cross-section through a thickness according to at least one example embodiment.

In at least one example embodiment, the cross-section through the thickness of the recessed portion 1600A slightly tapers and squares-off on a side 1604B of the cross-section, forming half of the capsule-shaped nicotine gum piece.

In at least one alternative example embodiment, the cross-section tapers to a rounded point (similar to the point 1104A in FIG. 11B) on each side 1604B of the cross-section. In at least one alternative example embodiment, the point is not a rounded point.

In at least another example embodiment, a recessed portion of the plurality of recessed portions 520 defines another cross-sectional shape. In at least one example embodiment, the recessed portion has a semi-circular cross-sectional shape, a boomerang cross-sectional shape, a comma cross-sectional shape, a bowtie cross-sectional shape, or a bean/kidney cross-sectional shape, or any other cross-sectional shape.

In at least one example embodiment, a recessed portion of the plurality of recessed portions 520 has a non-uniform thickness (e.g., a pillow shape). In at least one alternative example embodiment, a recessed portion of the plurality of recessed portions 520 has a uniform thickness. In at least one example embodiment, the recessed portion does not have a single smallest dimension, or single thickness dimension (e.g., a wedge or a pillow shape).

FIG. 17 is a perspective view of a die used in an apparatus for forming nicotine gum according to at least one example embodiment.

In at least one example embodiment, the die 178 includes the plurality of recessed portions 182 and the plurality of non-recessed portions 404. In at least one example embodiment, the die 178 is the same as the die 178 in FIGS. 5-7 except that one segment 1712 of the plurality of non-recessed portions 404 does not define a boundary of two different recessed portions 182. Instead, one segment 1712 of the plurality of non-recessed portions 404 defines a boundary of one recessed portion 182 and another segment 1712 of the plurality of non-recessed portions 404 defines a boundary of an adjacent recessed portion 182. In at least one example embodiment, a gap recess 1716 separates the one segment 1712 from the other segment 1712.

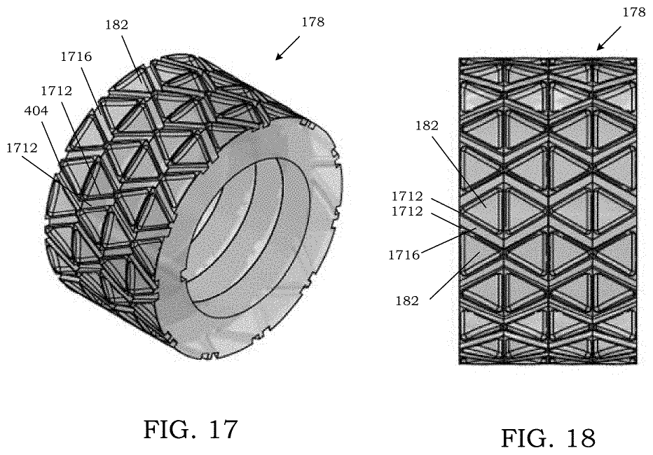

FIG. 18 is a front view of a die used in an apparatus for forming nicotine gum according to at least one example embodiment.

In at least one example embodiment, the gap recess 1716 extends a length of the die 178 and separates adjacent recessed portions 182 in a direction around a circumference of the die 178.

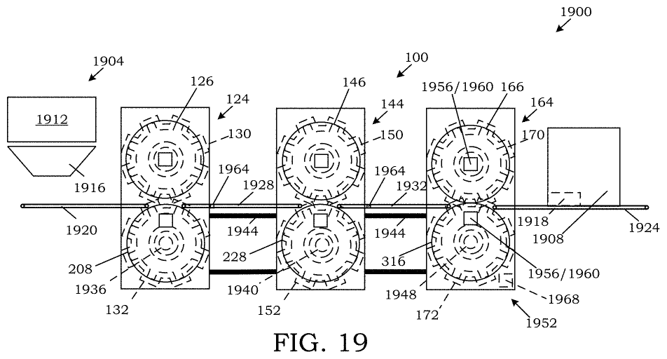

FIG. 19 is a schematic view of an apparatus for forming nicotine gum according to at least one example embodiment.

In at least one example embodiment, a gum forming system 1900 includes the apparatus 100. The gum forming system 1900 additionally includes a gum mixture supply system 1904 and a packaging assembly 1908.

In at least one example embodiment, the gum mixture supply system 1904 includes a mixer 1912 and a material feeder 1916. The mixer 1912 and the material feeder 1916 are positioned along a vertical axis with the mixer 1912 being positioned vertically above the material feeder 1916.

In at least one example embodiment, the mixer 1912 is configured to combine and mix the ingredients for the gum mixture. In at least one example embodiment, the mixer 1912 is the blending system described in U.S. Publication No. 2019/0321793 published Oct. 24, 2019, the disclosure of which is incorporated herein in its entirety by reference. The mixed gum mixture is transferred to the material feeder 1916. In at least one example embodiment, the material feeder 1916 is a nozzle-shaped housing that dispenses the gum mixture. In at least one example embodiment, the material feeder 1916 includes a hopper. In at least one example embodiment, the material feeder 1916 is the hopper and measuring system described in U.S. Pat. No. 10,399,712 to Longest et. al (filed Dec. 29, 2014), the disclosure of which is incorporated herein in its entirety by reference.

In at least one example embodiment, the packaging assembly 1908 is configured to package the gum pieces for distribution and sale. In at least one example embodiment, the packaging assembly 1908 is the apparatus for filling a plurality of containers with products as described in U.S. patent application Ser. No. 17/179,610 filed Feb. 19, 2021, the disclosure of which is incorporated herein in its entirety by reference.

In at least one example embodiment, the packaging assembly 1908 includes a shaker 1918 that separates the plurality of gum pieces from the scored sheet. The shaker 1918 is configured to vibrate the scored sheet to break the plurality of gum pieces along the connectors 1008 and separate the plurality of gum pieces. In at least one alternative example embodiment, an operator separates the plurality of gum pieces from the scored sheet. In at least one alternative example embodiment, the packaging assembly 1908 receives individual gum pieces and sorts and packages the plurality of gum pieces.

In at least one example embodiment, the system 1900 includes a supply conveyor 1920, discharge conveyor 1924, a first conveyor 1928, and a second conveyor 1932 in place of plates for transporting the nicotine gum material, the first pressed sheet, the second pressed sheet, and the plurality of gum pieces. In at least one example embodiment, the conveyors 1920, 1924, 1928, 1932 are belt and pulley conveyors.

In at least one example embodiment, the first pair of drums 124 in the first section 110 are driven by a motor 1936. The motor 1936 engages with the first lower gear assembly 132 to drive rotation of the first lower gear assembly 132. Rotation of the first lower gear assembly 132 rotates the first lower drum 208 and drives rotation of the first upper gear assembly 130. Rotation of the first upper gear assembly 130 rotates the first upper drum 126. In at least one alternative embodiment, the motor 1936 engages the first upper gear assembly 130, having a reverse effect.

In at least one example embodiment, the second pair of drums 144 in the second section 114 are driven by a motor 1940. The motor 1940 engages with the second lower gear assembly 152 to drive rotation of the second lower gear assembly 152. Rotation of the second lower gear assembly 152 rotates the second lower drum 228 and drives rotation of the second upper gear assembly 150. Rotation of the second upper gear assembly 150 rotates the second upper drum 146. In at least one alternative embodiment, the motor 1940 engages the second upper gear assembly 150, having a reverse effect.

In at least one example alternative embodiment, the second upper gear assembly 150, the second lower gear assembly 152, or a combination thereof, is engaged with the first upper gear assembly 130, the first lower gear assembly 132, or a combination thereof, of the first section 110 and drives, or is driven by, the first upper gear assembly 130, the first lower gear assembly 132, or a combination thereof. In at least one example embodiment, the second lower gear assembly 152 is engaged with the first lower gear assembly 132 through a belt, chain, or pulley 1944.

In at least one example embodiment, the third pair of drums 164 in the third section 118 is driven by a motor 1948. The motor 1948 engages with the third lower gear assembly 172 to drive rotation of the third lower gear assembly 172. Rotation of the third lower gear assembly 172 rotates the third lower drum 316 and drives rotation of the third upper gear assembly 170. Rotation of the third upper gear assembly 170 rotates the third upper drum 166. In at least one alternative embodiment, the motor 1948 engages with and drives the third upper gear assembly 170, having a reverse effect.

In at least one example alternative embodiment, the third upper gear assembly 170, the third lower gear assembly 172, or a combination thereof, is engaged with the first upper gear assembly 130, the first lower gear assembly 132, the second upper gear assembly 150, the second lower gear assembly 152, or a combination thereof, and drives, or is driven by, the first upper gear assembly 130, the first lower gear assembly 132, the second upper gear assembly 150, the second lower gear assembly 152, or a combination thereof. In at least one example embodiment, the third lower gear assembly 172 is engaged with the first lower gear assembly 132 and the second lower gear assembly 152 through the belt, chain, or pulley 1944.

In at least one example embodiment, the system 1900 includes a control system 1952. In at least one example embodiment the control system 1952 includes a heater 1956, a cooler 1960, or a combination thereof, a sensor 1964, and a controller 1968. In at least one example embodiment, the control system 1952 is configured to regulate a temperature of the first pair of drums 124, the second pair of drums 144, and the third pair of drums 164. In at least one example embodiment, the heater 1956, cooler 1960, or combination thereof, is disposed in the first upper drum 126, the first lower drum 208, the second upper drum 146, the second lower drum 228, the third upper drum 166, the third lower drum 316, or a combination thereof.

In at least one example embodiment, the heater 1956 is configured to increase a surface temperature of the drum in which it is disposed. The increased surface temperature transfers heat to the gum mixture, the first pressed sheet, or the second pressed sheet. Increasing the temperature of the gum mixture, the first pressed sheet, or the second pressed sheet may soften the gum mixture, the first pressed sheet, or the second pressed sheet, making the gum mixture, the first pressed sheet, or the second pressed sheet easier to form.

In at least one example embodiment, the cooler 1960 is configured to decrease a surface temperature of the drum in which it is disposed. The decreased surface temperature transfers heat away from the gum mixture, the first pressed sheet, or the second pressed sheet. The gum mixture, the first pressed sheet, or the second pressed sheet may increase in temperature during pressing, forming, or a combination thereof. Decreasing the temperature of the gum mixture, the first pressed sheet, or the second pressed sheet may regulate the temperature of the gum mixture, the first pressed sheet, or the second pressed sheet. Decreasing the temperature of the gum mixture, the first pressed sheet, or the second pressed sheet may make the gum mixture, the first pressed sheet, or the second pressed sheet harder or more rigid.

In at least one example embodiment, the sensor 1964 is disposed in the first conveyor 1928, the second conveyor 1932, the first upper drum 126, the first lower drum 208, the second upper drum 146, the second lower drum 228, the third upper drum 166, the third lower drum 316, or a combination thereof. The sensor 1964 senses a temperature of the gum mixture, the first pressed sheet, or the second pressed sheet.

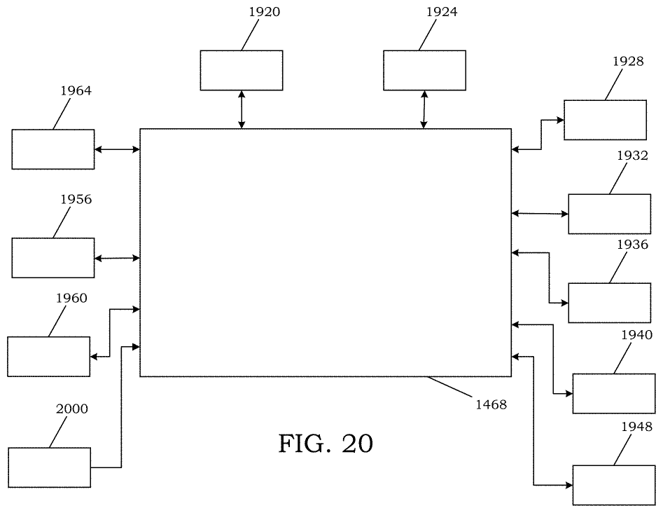

FIG. 20 is a schematic diagram of a control system for an apparatus for forming nicotine gum according to at least one example embodiment.

In at least one example embodiment, the controller 1968 is in communication with the heater 1956, the cooler 1960, and the sensor 1964. The controller 1968 actuates or de-activates the heater 1956 and the cooler 1960 based on outputs from the sensor 1964.

In at least one example embodiment, the controller 1968 selectively actuates the heater 1956 based on an output of the sensor 1964 indicating that a temperature is lower than a lower heating temperature threshold. The lower heating temperature threshold may range from about 1° C. to about 5° C. less than a set point temperature (e.g., the set point temperature may range from about 15° C. to about 50° C., or about 25° C.). The controller 1968 selectively shuts off the heater 1956 based on the output of the sensor 1964 indicating that the temperature is higher than an upper heating temperature threshold. The upper heating temperature threshold may range from about 1° C. to about 5° C. greater than the set point temperature.

In at least one alternative example embodiment, the controller 1968 selectively actuates and shuts off the heater 1956 based on a running state of one or more of the conveyors 1920, 1924, 1928, 1932, one or more of the motors 1936, 1940, and 1948, or a combination thereof. The controller 1968 receives a signal to start one of the conveyors 1920, 1924, 1928, 1932, one of the motors 1936, 1940, 1948, or a combination thereof, and starts the heater 1956 based on the signal.

In at least one example embodiment, the controller 1968 sets a temperature or heating level of the heater 1956. The controller 1968 accesses a look-up table (LUT) stored in a memory of the controller 1968. The LUT maps an input to an output (for example, an output of the sensor to a control value input of the heater). The LUT is derived as a design parameter through empirical study. The controller 1968 obtains a control value for the heater 1956 based on the output of the sensor 1964.

In at least one alternative example embodiment, the controller 1968 sets the temperature or heating level of the heater 1956 based on the output of the sensor 1964. In at least one example embodiment, the controller 1968 compares the output of the sensor 1964 with one or more temperature thresholds to determine a temperature of the heater 1956. The temperature thresholds indicate increasing distance from the lower heating temperature threshold. The controller 1968 sets the temperature or heating level to an increased heating position based on the increasing distance from the lower heating temperature threshold.

In at least one example embodiment, the controller 1968 selectively actuates the cooler 1960 based on an output of the sensor 1964 indicating that a temperature is higher than an upper cooling temperature threshold. The upper cooling temperature may range from about 1° C. to about 5° C. higher than a set point temperature (e.g., the set point temperature may range from about 15° C. to about 50° C., or about 25° C.). The controller 1968 selectively shuts off the cooler 1960 based on the output of the sensor 1964 indicating that the temperature is lower than a lower cooling temperature threshold. The lower cooling temperature threshold may range from about 1° C. to about 5° C. lower than the set point temperature.

In at least one alternative example embodiment, the controller 1968 selectively actuates and shuts off the cooler 1960 based on a running state of one or more of the conveyors 1920, 1924, 1928, 1932, one or more of the motors 1936, 1940, and 1948, or a combination thereof. The controller 1968 receives a signal to start one of the conveyors 1920, 1924, 1928, 1932, one of the motors 1936, 1940, 1948, or a combination thereof, and starts the cooler 1960 based on the signal.

In at least one example embodiment, the controller 1968 sets a temperature or cooling level of the cooler 1960. The controller 1968 accesses a look-up table (LUT) stored in a memory of the controller 1968. The LUT maps an input to an output (for example, an output of the sensor to a control value input of the cooler). The LUT is derived as a design parameter through empirical study. The controller 1968 obtains a control value for the cooler 1960 based on the output of the sensor 1964.

In at least one alternative example embodiment, the controller 1968 sets the temperature or cooling level of the cooler 1960 based on the output of the sensor 1964. In at least one example embodiment, the controller 1968 compares the output of the sensor 1964 with one or more temperature thresholds to determine a temperature or cooling level of the cooler 1960. The temperature thresholds indicate increasing distance from the upper cooling temperature threshold. The controller 1968 sets the temperature or cooling level to an increased cooling position based on the increasing distance from the upper cooling temperature threshold.

In at least one example embodiment, the controller 1968 is in communication with a user control 2000, the supply conveyor 1920, the discharge conveyor 1924, the first conveyor 1928, and the second conveyor 1932. The controller 1968 selectively activates or shuts down the conveyors 1920, 1924, 1928, 1932 based on an output signal from the user control 2000. In at least one example embodiment, the controller 1968 controls a speed of one or more of the conveyors 1920, 1924, 1928, 1932 based on an output signal from the user control 2000, speeds of the drums 126, 132, 146, 152, 166, 172, or a combination thereof. In at least one example embodiment, the speeds of the drums 126, 132, 146, 152, 166, 172 are determined from a speed of the motors 1936, 1940, 1948.

In at least one example embodiment, the user control 2000 provides a signal to start one or more of the conveyors 1920, 1924, 1928, 1932. In at least one example embodiment, the user control 2000 is a switch (for example, a toggle switch), one or more buttons, or a combination thereof. The controller 1968 receives the output from the user control 2000 and, based on the output, starts the conveyors 1920, 1924, 1928, 1932.

In at least one example embodiment, the conveyors 1920, 1924, 1928, 1932 are each operated by a motor (not shown). The controller 1968 starts the conveyors 1920, 1924, 1928, 1932 by starting each of the motors. In at least one alternative example embodiment, the conveyors 1920, 1924, 1928, 1932 are operated by a single motor (not shown). The controller 1968 starts the conveyors 1920, 1924, 1928, 1932 by starting the motor.

In at least one example embodiment, the controller 1968 is in communication with the motor 1936 rotating the first pair of drums 124, the motor 1940 rotating the second pair of drums 144, and the motor 1948 rotating the third pair of drums 164. The controller 1968 selectively activates or shuts down rotation of the first pair of drums 124, the second pair of drums 144, and the third pair of drums 164 by selectively starting or stopping the motors 1936, 1940, and 1948, respectively. In at least one example embodiment, the controller 1968 controls a speed of the first pair of drums 124, the second pair of drums 144, and the third pair of drums 164, or a combination thereof by controlling a speed of the motors 1936, 1940, and 1948, respectively.

In at least one example embodiment, the user control 2000 (for example a switch, one or more buttons, or a combination thereof) provides a signal to start the first pair of drums 124, the second pair of drums 144, the third pair of drums 164, or a combination thereof. The controller 1968 receives the output from the user control 2000 and, based on the output, starts and sets a speed of the first motor 1936, the second motor 1940, the third motor 1948, or a combination thereof.

The controller 1968, according to one or more example embodiments, may be implemented using hardware, or a combination of hardware and software. Hardware may be implemented using processing or control circuitry such as, but not limited to, one or more processors, one or more Central Processing Units (CPUs), one or more microcontrollers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUs), one or more microprocessors, one or more Application Specific Integrated Circuits (ASICs), or any other device or devices capable of responding to and executing instructions in a defined manner.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform (e.g., execute) the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiment described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiment, or they may be known devices that are altered and/or modified for the purposes of example embodiment.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as one computer processing device; however, one skilled in the art will appreciate that a hardware device may include multiple processing elements and multiple types of processing elements. For example, a hardware device may include multiple processors or a processor and a controller. In addition, other processing configurations are possible, such as parallel processors.

Software may include a computer program, program code, instructions, or some combination thereof, for independently or collectively instructing or configuring a hardware device to operate as desired. The computer program and/or program code may include program or computer-readable instructions, software modules, data files, data structures, and/or the like, capable of being implemented by one or more hardware devices, such as one or more of the hardware devices mentioned above. Examples of program code include both machine code produced by a compiler and higher level program code that is executed using an interpreter.

Software and/or data may be embodied permanently or temporarily in any type of machine, element, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media or memory.

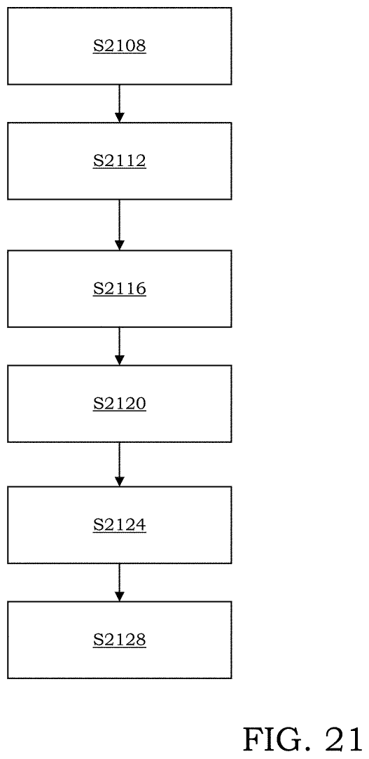

FIG. 21 is a flow chart of a method of forming nicotine gum according to at least one example embodiment.

In at least one example embodiment, at 52108 the first pair of drums 124 begins rotation and a gum mixture is fed into the first pair of drums 124. In at least one example embodiment, the first upper drum 126 rotates in a counterclockwise direction, while the first lower drum 208 rotates in a clockwise direction. In at least one example embodiment, the first upper drum 126 rotates at a first speed and the first lower drum 208 rotates at a second speed, such that there is consistent material transfer through the first upper drum 126 and first lower drum 208. In at least one example embodiment, the first upper drum 126 and the first lower drum 208 are manually rotated, automatically rotated by a motor, or a combination thereof.

At S2112, the gum mixture is pressed within the first pair of drums 124. In at least one example embodiment, the gum mixture is fed into the gap 304 separating the first upper drum 126 from the first lower drum 208. Rotation of the first upper drum 126 and first lower drum 208 draws the gum mixture through the gap 304. The gum mixture is pressed between the first upper drum 126 and first lower drum 208 into the first pressed sheet. As previously stated, in at least one example embodiment, the first pressed sheet includes a thickness ranging from about 4 mm to about 20 mm (e.g., about 8 mm to about 11 mm or about 9.5 mm to about 10 mm). The first pressed sheet exits the gap 304 and first pair of drums 124 onto the first plate 240 or first conveyor 1428.

At S2116, the second pair of drums 144 begin rotation and the first pressed sheet is fed into the second pair of drums 144. In at least one example embodiment, the first pressed sheet is transferred along the first plate 240, or first conveyor 1428, from the first pair of drums 124 to the second pair of drums 144. The second upper drum 146 and second lower drum 228 of the second pair of drums 144 rotate. In at least one example embodiment, the second upper drum 146 rotates in a counterclockwise direction, while the second lower drum 228 rotates in a clockwise direction.

In at least one example embodiment, the second upper drum 146 rotates at a first speed and the second lower drum 228 rotates at a second speed, such that there is consistent material transfer through the second upper drum 146 and second lower drum 228. In at least one example embodiment, the second upper drum 146 and the second lower drum 228 are manually rotated, automatically rotated by a motor, or a combination thereof.

At S2120, the first pressed sheet is further pressed to a second thickness by the second pair of drums 144. In at least one example embodiment, the first pressed sheet is fed into the gap 308 separating the second upper drum 146 from the second lower drum 228. Rotation of the second upper drum 146 and second lower drum 228 draws the first pressed sheet through the gap 308. The first pressed sheet is pressed between the second upper drum 146 and second lower drum 228 into the second pressed sheet. As previously stated, in at least one example embodiment, the second pressed sheet has a thickness ranging from about 2 mm to about 18 mm (e.g., about 6 mm to about 9 mm or about 7.5 mm to about 8 mm). The second pressed sheet exits the gap 308 and second pair of drums 144 onto the second plate 252.

At S2124, the third pair of drums 164 begin rotation and the second pressed sheet is fed into the third pair of drums 164. In at least one example embodiment, the second pressed sheet is transferred along the second plate 252, or second conveyor 1432, from the second pair of drums 144 to the third pair of drums 164. The third upper drum 166 and third lower drum 316 of the third pair of drums 164 rotate. In at least one example embodiment, the third upper drum 166 rotates in a counterclockwise direction, while the third lower drum 316 rotates in a clockwise direction.

In at least one example embodiment, the third upper drum 166 rotates at a first speed and the third lower drum 316 rotates at a second speed, such that there is consistent material transfer through the third upper drum 166 and third lower drum 316. In at least one example embodiment, the third upper drum 166 and the third lower drum 316 are manually rotated, automatically rotated by a motor, or a combination thereof.

At S2128, the third pair of drums 164 form the plurality of gum pieces. In at least one example embodiment, the third pair of drums 164 form a scored sheet having the plurality of gum pieces. In at least one alternative example embodiment, the third pair of drums 164 form the plurality of gum pieces as individual, separate gum pieces.

In at least one example embodiment, the second pressed sheet is fed into the gap 336 separating the third upper drum 166 from the third lower drum 316. Rotation of the third upper drum 166 and third lower drum 316 draws the second pressed sheet through the gap 336. The second pressed sheet is formed between the third upper drum 166 and third lower drum 316.

In at least one example embodiment, the second pressed sheet is scored by the third upper drum 166 and the third lower drum 316, forming a scored sheet. The scored sheet defines the plurality of gum pieces. In at least one example embodiment, the plurality of gum pieces remain connected in the scored sheet. In at least one alternative example embodiment, the second pressed sheet is cut by the third upper drum 166 and the third lower drum 316 into the individual, separate, plurality of gum pieces. The plurality of gum pieces exits the gap 336 and third pair of drums 164.

While some example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

We claim:

1. An apparatus for forming a nicotine gum, the apparatus comprising:
a first pair of drums configured to receive and press a nicotine gum mixture to a first thickness so as to form a first pressed sheet; and
a second pair of drums configured to receive and score the first pressed sheet so as to form a scored sheet including a plurality of gum pieces, the second pair of drums including,
a first drum including a first die, the first die including a first pattern, and
a second drum including a second die, the second die including a second pattern, the second pattern being an mirror image of the first pattern, and the first drum and the second drum spaced such that as the first pressed sheet passes between the first drum and the second drum, the first die and the second die at least score the first pressed sheet.

2. The apparatus of claim 1, wherein the first die defines a first plurality of recessed portions.

3. The apparatus of claim 2, wherein the second die defines a second plurality of recessed portions corresponding to the first plurality of recessed portions.

4. The apparatus of claim 3, wherein the second pair of drums is configured to align a first plurality of non-recessed portions on the first die with a second plurality of non-recessed portions on the second die so as to at least score the first pressed sheet into the plurality of gum pieces.

5. The apparatus of claim 3, wherein
the first drum of the second pair of drums is configured to rotate at a first speed,
the second drum of the second pair of drums is configured to rotate at a second speed, and
the first speed and the second speed are established to facilitate consistent material transfer, such that when a first plurality of non-recessed portions on the first die is aligned with a second plurality of non-recessed portions on the second die, the non-recessed portions at least score the first pressed sheet.

6. The apparatus of claim 2, wherein each of the first plurality of recessed portions has a triangular cross-section along an upper surface of a non-recessed portion of the first die.

7. The apparatus of claim 2, wherein a first segment of a non-recessed portion on the first die defines one side of each of a pair of adjacent recessed portions.

8. The apparatus of claim 1, wherein the second pair of drums is downstream of the first pair of drums.

9. The apparatus of claim 8, further comprising:
a third pair of drums between the second pair of drums and the first pair of drums, the third pair of drums being configured to receive and press the first pressed sheet to a second uniform thickness.

10. The apparatus of claim 1, further comprising:
a heater configured to heat the first drum of the second pair of drums, the second drum of the second pair of drums, or a combination thereof, so as to heat the first pressed sheet.

11. A method for forming a nicotine gum, the method comprising:
passing a sheet of a gum mixture through a first pair of drums;
pressing the sheet between the first pair of drums to a first thickness so as to form a first pressed sheet; and
passing the first pressed sheet through a second pair of drums, a first drum of the second pair of drums including a first die and a second drum of the second pair of drums including a second die, the first die including a first pattern of recessed portions and the second die including a second pattern of recessed portions, the first pattern of recessed portions being a mirror image of the second pattern of recessed portions so as to at least score the first pressed sheet and form a scored sheet including a plurality of gum pieces.

12. The method of claim 11, wherein the second pair of drums is downstream of the first pair of drums.

13. The method of claim 12, further comprising:
passing the first pressed sheet though a third pair of drums, the third pair of drums being between the second pair of drums and the first pair of drums so as to press the first pressed sheet to a second uniform thickness.

14. The method of claim 13, wherein the second uniform thickness ranges from 2 mm to 18 mm.

15. The method of claim 11, further comprising:
regulating a temperature of the first pair of drums and the second pair of drums.

16. The method of claim 15, wherein the regulating a temperature comprises:
heating the second pair of drums so as to increase a temperature of the first pressed sheet.

17. The method of claim 15, wherein the regulating a temperature comprises:
cooling the second pair of drums so as to reduce a temperature of the first pressed sheet.

18. The method of claim 11, wherein the passing the first pressed sheet through a second pair of drums comprises:
aligning a first non-recessed portion on the first die with a second non-recessed portion on the second die so as to at least score the first pressed sheet.

19. The method of claim 11, wherein the first thickness ranges from 4 mm to 20 mm.

20. The method of claim 11, wherein the passing the first pressed sheet through a second pair of drums comprises:
rotating the first drum of the second pair of drums at a first speed;
rotating the second drum of the second pair of drums at a second speed, the first speed being the same as the second speed; and
aligning a non-recessed portion of the first pattern of recessed portions on the first die with a non-recessed portion of the second pattern of recessed portions on the second die so as to at least score the first pressed sheet and form the plurality of gum pieces.

21. An apparatus for forming a nicotine gum, the apparatus comprising:
a first pair of drums configured to receive and press a nicotine gum mixture to a first thickness so as to form a first pressed sheet; and
a second pair of drums configured to receive and cut the first pressed sheet so as to form a plurality of gum pieces, the second pair of drums including,
a first drum including a first die, the first die including a first pattern, and
a second drum including a second die, the second die including a second pattern, the second pattern being an mirror image of the first pattern, and the first drum and the second drum spaced such that as the first pressed sheet passes between the first drum and the second drum, the first die and the second die cut the plurality of gum pieces.
